(19) United States
(12) Patent Application Publication
(10) Pub. No.: US 2018/0072970 A1
(43) Pub. Date: Mar. 15, 2018

(54) STABLE VIOLET-BLUE TO BLUE IMIDAZOLIUM COMPOUNDS

(71) Applicant: The Procter & Gamble Company, Cincinnati, OH (US)

(72) Inventors: Bryan Patrick MURPHY, Loveland, OH (US); Guiru ZHANG, Lebanon, OH (US); Jielu ZHAO, Mason, OH (US)

(21) Appl. No.: 15/263,912

(22) Filed: Sep. 13, 2016

(51) Int. Cl. C11D 3/42; C07D 403/12; C09B 44/16; C07D 498/06; C09B 69/10; C08G 73/02

(52) U.S. Cl. CPC C11D 3/42; C07D 403/12; C08G 73/0206; C07D 498/06; C09B 69/106; C09B 44/16

(57) ABSTRACT

Disclosed are novel stable violet-blue to blue imidazolium azo compounds that have a simplified chromophore and high relative solubility in aqueous systems, and that are stable under the conditions of use and storage. The compounds are useful for dyeing fibers such as fabrics and hair, as fabric hueing agents, and as dyes for other keratinaceous materials. Also disclosed are laundry care compositions comprising the imidazolium azo compounds and methods of using the compositions.

### TECHNICAL FIELD OF THE INVENTION

The present invention relates to novel stable violet-blue to blue imidazolium azo compounds that have a simplified chromophore and high relative solubility in aqueous systems, and that are stable under the conditions of use and storage. These are useful for dyeing fibers such as fabrics and hair, as fabric hueing agents, and as dyes for other keratinaceous materials. This invention also relates to laundry care compositions comprising the imidazolium azo compounds that may serve as bluing agents, and methods of using the same.

### BACKGROUND OF THE INVENTION

In general, violet-blue to blue colors used for dyeing or hueing agents for materials such as plastics, fabrics, and hair are either fused polycyclics such as substituted phenoxazines and anthraquinones, carbocyclic azo dyes in which the acceptor half of the molecule is highly substituted with electron-withdrawing groups used to shift the color into the desired range, bis(azo) compounds that extend conjugation, or azo dyes that have a carbocyclic donor portion and a thiazolium or thiazole (substituted with electron-withdrawing groups such as cyano or nitro) acceptor portion. Considering the donor-acceptor approach of color design, the well-known tactics are to include powerful election-withdrawing groups in the acceptor end of the molecule, thus pulling electron density towards the acceptor portion, driving the color to higher absorbance wavelengths. Although it can give the desired color shift, it can decrease the stability of the dyes, particularly when the acceptor end is a heterocycle such as imidazolium or thiazolium.

Although these well-known and well-accepted aforementioned classes of dyes provide the violet-blue to blue colors desired, each of these classes has at least one flaw that makes it inappropriate for dyeing at alkaline pH in highly aqueous systems. (More aqueous dyebaths are desired to decrease the environmental impact relative to dyebaths that use high levels of organic solvents.) The solubility, fastness properties, or stability of current materials may be insufficient for the applications. In cases in which solubility, fastness, and stability are sufficient, the color cannot have the required blue hue. Specifically in applications such as hair dyeing and fabric hueing or dyeing, this is an important outage.

Thiazolium azo and thiazole azo compounds in particular provide brilliant colors with high intensity. However, it is well-known that particularly for the vibrant and water soluble thiazolium dyes, stability is poor. Common commercial imidazolium dyes (analogs of the thiazolium dyes) can give brilliant and stable orange to violet-red colors. However, there are no good examples of blue imidazolium dyes, most likely because using the common approach of adding electron-withdrawing groups to the acceptor (imidazolium) end of the molecule results in materials that are highly unstable, especially at alkaline pH.

Baumann and Dehnert described generating a violet imidazolium azo dye (in Chimia 15, 1961, 163-168) however they could not obtain blues, and Abbot, et al. (in J. Phys. Chem. J. Phys. Chem. A 2013, 117, 1853-1871) demonstrated the inherent instability of thiazolium compounds under basic conditions, making them unsuitable for applications for which neutral to alkaline pH is necessary.

### SUMMARY OF THE INVENTION

The present invention relates to stable violet-blue to blue chromophores based on a combination of the imidazolium azo core with electron-donating groups in the donor portion of the molecule that previously has not been exploited to produce blue colors. These are useful for dyeing fibers such as fabrics and hair, as fabric hueing agents, and as dyes for other keratinaceous materials. Unlike completely carbocycle-based azo dyes in the blue-violet to blue range, these materials have high solubility in aqueous systems. Unlike charged heterocyclic blue dyes, and uncharged heterocyclic blue dyes that are highly substituted with electron-withdrawing groups on the acceptor side of the dye, they are surprisingly stable under conditions of use and storage, including in the presence of oxidants such as hydrogen peroxide. The invention also relates to laundry care compositions comprising the imidazolium azo compounds that may serve as bluing agents, and methods of using the same.

### DETAILED DESCRIPTION OF THE INVENTION

While the specification concludes with claims which particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. When more than one composition is used during a treatment, as in mixing of the components, the total weight to be considered is the total weight of all the compositions applied on the substrate simultaneously unless otherwise specified. The term “weight percent” may be denoted as “wt %” herein.

As used herein, “cellulosic substrates” are intended to include any substrate which comprises at least a majority by weight of cellulose. Cellulose may be found in wood, cotton, linen, jute, and hemp. Cellulosic substrates may be in the form of powders, fibers, pulp and articles formed from powders, fibers and pulp. Cellulosic fibers, include, without limitation, cotton, rayon (regenerated cellulose), acetate (cellulose acetate), triacetate (cellulose triacetate), and mixtures thereof. Articles formed from cellulosic fibers include textile articles such as fabrics. Articles formed from pulp include paper.

As used herein, the term “chromophore” means the part of the direct dye compound responsible for its color.

As used herein, the term “direct dye compound” means a dye used in a process in which dye molecules are attracted by physical forces at the molecular level to a textile or substrate such as the hair. As opposed to oxidative dyes, there is no chemical reaction required to form the chromophore. Additionally, there is no covalent bond formation between the direct dye and the substrate, as is the case for reactive dyes. The direct dye compound does not undergo a chemical transformation before and after the dyeing process.

As used herein, the term “laundry care composition” includes, unless otherwise indicated, granular, powder, liquid, gel, paste, unit dose, bar form and/or flake type washing agents and/or fabric treatment compositions.

The compositions of the present invention comprise one or more stable violet blue to blue direct dyes based on the imidazolium azo core, and optionally other dye materials.

With regards to the compounds described herein, numerous tautomeric compounds may be involved. Thus, for example, 2-mercaptopyridine (I) may exist under known conditions in the pyridine-2-thione tautomer form (II).

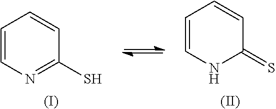

It is to be understood that when this development refers to a particular structure, all of the reasonable additional tautomeric structures are included. In the art, tautomeric structures are frequently represented by one single structure and the method described herein follows this general practice.

It is also understood that within the scope of this invention, E, Z isomers may be involved. Thus, for example, (E)-diphenyldiazene (III) converts under known conditions to (Z)-diphenyldiazene (IV), which is also reversible.

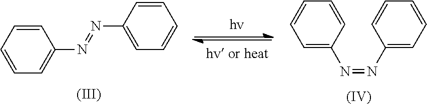

It is to be understood that when this development refers to a particular structure, all of the reasonable additional E, Z isomers are included.

When a salt of Formula (Xa) or Formula (Xb) contains a cationic moiety, anionic counterions include, for example, D,L-malate, L-malate, D-malate, chloride, bromide, citrate, acetate, lactate, succinate, tartrate, phosphate, hemisulfate, sulfate, methylsulfate, trifluoroacetate, iodide, and mixtures thereof. When a salt of Formula (Xa) or Formula (Xb) contains an anionic moiety, cationic counterions include, for example, ammonium, substituted ammonium salts (e.g., monoethanolammonium, diethanolammonium, triethanolammonium), sodium, potassium, and mixtures thereof.

### I. Stable Blue-Violet to Blue Imidazolium Compounds

Described herein are unique blue-violet to violet imidazolium chromophores that are stable under conditions of use for hair dyeing and fabric dyeing and hueing and that are storage stable.

In some embodiments, the compound is of Formula Xa, or a tautomer or salt thereof

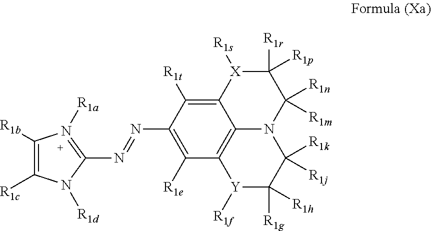

wherein
(i) R_{1g}, R₁ₕ, R₁ⱼ, R₁ₖ, R₁ₘ, R₁ₙ, R₁ₚ and R₁ᵣ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, nitro, nitroso, cyano, carboxyl, a heterocyclic moiety, thioether, thiol with a linker group, alkylsulfonate, alkylsulfate, carboxylalkyl, acrylamide or substituted acrylamides with or without a linker group, vinylsulfone with or without a linker group, sulfonyl ethyl sulfate with or without a linker group, halo-s-triazines with or without a linker group, halopyrimidines with or without a linker group, haloquinoxalines with or without a linker group, or are attached to a polymer backbone through a linker;
(ii) R_{1b}, R_{1c}, R₁ₑ and R₁ₜ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iii) R_{1f} and R₁ₛ are each independently hydrogen, alkyl, halogen substituted alkyl, alkenyl, alkynyl, aryl, hydroxyalkyl, aminoalkyl, alkyl group carrying a quaternary ammonium cation, acyl, a heterocyclic moiety, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iv) R₁ₐ and R_{1d} are each independently alkyl, halogen substituted alkyl, alkenyl, alkynyl, aryl, hydroxyalkyl, aminoalkyl, alkyl group carrying a quaternary ammonium cation, alkyl group carrying a quaternary ammonium cation, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(v) X and Y are each independently an oxygen or a nitrogen atom and when X or Y is a nitrogen atom the other can be a carbon atom; wherein in the case where X and/or Y is oxygen atom, the corresponding group attached to the oxygen atom, R_{1f} and/or R₁ₛ, ceases to exist.

In some embodiments for compounds of Formula (Xa), at least one of X and Y is an oxygen atom, in some embodiments both X and Y are oxygen atoms. In some embodiments, R₁ₐ, R_{1d}, R_{1f} and R₁ₛ are each independently alkenyl, alkyl, aminoalkyl or alkyl group carrying a quaternary ammonium cation, in some embodiments the compound of Formula (Xa) is attached to a polymer through one or more of R₁ₐ, R_{1d}, R_{1f} and R₁ₛ. In some embodiments, R_{1b}, R_{1c}, R₁ₑ, R_{1f}, R_{1g}, R₁ₕ, R₁ⱼ, R₁ₖ, R₁ₘ, R₁ₙ, R₁ₚ and R₁ᵣ are each independently hydrogen or alkyl.

In other embodiments, the compound is of Formula (Xb), or a tautomer or salt thereof;

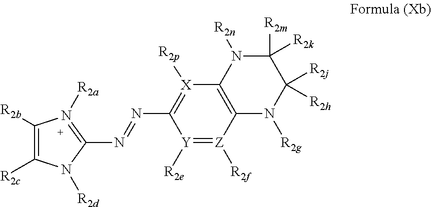

wherein
(i) R₂ₕ, R₂ⱼ, R₂ₖ and R₂ₘ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, nitro, nitroso, cyano, carboxyl, a heterocyclic moiety, thioether, thiol with a linker group, alkylsulfonate, alkylsulfate, carboxylalkyl, acrylamide or substituted acrylamides with or without a linker group, vinylsulfone with or without a linker group, sulfonyl ethyl sulfate with or without a linker group, halo-s-triazines with or without a linker group, halopyrimidines with or without a linker group, haloquinoxalines with or without a linker group, or are attached to a polymer backbone through a linker;
(ii) R_{2b}, R_{2c}, R₂ₑ, R_{2f} and R₂ₚ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iii) R_{2g} and R₂ₙ are each independently hydrogen, alkyl, halogen substituted alkyl, alkenyl, alkynyl, aryl, hydroxyalkyl, aminoalkyl, alkyl group carrying a quaternary ammonium cation, acyl, a heterocyclic moiety, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iv) R₂ₐ and R_{2d} are each independently alkyl, halogen substituted alkyl, alkenyl, alkynyl, aryl, hydroxyalkyl, aminoalkyl, alkyl group carrying a quaternary ammonium cation, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(v) X, Y and Z are each independently a carbon or a nitrogen atom; wherein the total number of nitrogen atoms among X, Y and Z equals to 0 or 1; the total number of carbon atoms among X, Y and Z equals to 2 or 3; and, in the case where one of X, Y, Z is a nitrogen atom, the corresponding group attached to the nitrogen atom, one of R₂ₑ, R_{2f} or R₂ₚ, ceases to exist.

In some embodiments for compounds of Formula (Xb), R_{2g} and R₂ₙ are each independently hydrogen, alkenyl, alkyl, aminoalkyl or an alkyl group carrying a quaternary ammonium cation; in some embodiments R_{2g} is hydrogen, alkenyl or alkyl and R₂ₙ is alkenyl or alkyl.

In some embodiments, R₂ₐ and R_{2d} are each independently alkenyl, alkyl, aminoalkyl or alkyl group carrying a quaternary ammonium cation; in some embodiments R₂ₐ is alkyl and R_{2d} is aminoalkyl or an alkyl group carrying a quaternary ammonium cation; in some embodiments one or both of R₂ₐ and R_{2d} are an alkyl group carrying a quaternary ammonium cation.

In some embodiments, the compound of Formula (Xb) is attached to a polymer backbone through one of R₂ₐ, R_{2d}, R_{2g} or R₂ₙ.

In some embodiments, R_{2b}, R_{2c}, R₂ₑ, R_{2f} and R₂ₚ are each independently hydrogen or alkyl; in some embodiments R_{2b}, R_{2c}, R₂ₑ, R_{2f} and R₂ₚ are all hydrogen.

In some embodiments, R₂ₕ, R₂ⱼ, R₂ₖ and R₂ₘ are each independently hydrogen and alkyl; in some embodiments, R₂ₕ, R₂ⱼ, R₂ₖ and R₂ₘ are hydrogen.

In some embodiments, at least two of X, Y and Z are carbon; in some embodiments X, Y and Z are all carbon.

In embodiments where the compound of Formula (Xa) or (Xb) is linked to a polymer, the polymer may be selected from any of a variety of materials. Represented polymers include, but are not limited to, linear polyethyleneimines; branched polyethyleneimines consisting of primary, secondary and tertiary amine groups; polyallylamine hydrochloride; homopolymers or copolymers derived from acrylic or methacrylic esters or amides; copolymers of polystyrene sulfonate (PSS) and poly(4-styrenesulfonic acid-co-maleic acid); peptides, proteins; and the like.

In some embodiments when the compound of Formula (Xa) or (Xb) is linked to a polymer, the linking group can be of formula (L)

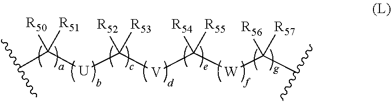

wherein
(i) L is covalently linked to the compound of Formula (Xa) or (Xb) either by its left-hand or right-hand side;
(ii) a, c, e and g are each independently an integer from 0 to 3, provided that the sum of a, c, e and g is greater than or equal to 2; b, d and f are each independently 0 or 1; and R₅₀, R₅₁, R₅₂, R₅₃, R₅₄, R₅₅, R₅₆ and R₅₇ are each independently hydrogen or C₁-C₂ alkyl group;
(iii) U is an aromatic ring, alkenyl or alkynyl moiety;
(iv) V is O, N or S; and
(v) W is a cyclic aliphatic ring.

### II. Laundry Care Compositions

Any of the bluing agents described in the present specification may be incorporated into laundry care compositions including but not limited to laundry detergents and fabric care compositions. The laundry care compositions including laundry detergents may be in solid or liquid form, including a gel form. Such compositions may comprise one or more of said bluing agents and a laundry care ingredient. The bluing agents may be added to substrates using a variety of application techniques. For instance, for application to cellulose-containing textile substrates, the bluing agent may be included as a component of a laundry detergent. Thus, application to a cellulose-containing textile substrate actually occurs when a consumer adds laundry detergent to a washing machine. The bluing agent may be present in the laundry detergent composition in an amount from about 0.00001% to about 15% by weight of the composition, from about 0.0001% to about 10% by weight of the composition, from about 0.0001% to about 5% by weight of the composition, from about 0.0001% to about 1% by weight of the composition, and even from about 0.0001% to about 0.5% by weight of the composition.

The laundry detergent composition typically comprises a surfactant in an amount sufficient to provide desired cleaning properties. In one aspect, the laundry detergent composition may comprise, based on total laundry detergent composition weight, from about 5% to about 90% of the surfactant, from about 5% to about 70% of the surfactant, or even from about 5% to about 40% of the surfactant. The surfactant may comprise anionic, nonionic, cationic, zwitterionic and/or amphoteric surfactants. In one aspect, the detergent composition comprises anionic surfactant, nonionic surfactant, or mixtures thereof.

Fabric care compositions are typically added in the rinse cycle, which is after the detergent solution has been used and replaced with the rinsing solution in typical laundering processes. The fabric care compositions disclosed herein may be comprise a rinse added fabric softening active and a suitable bluing agent as disclosed in the present specification.

The fabric care composition may comprise, based on total fabric care composition weight, from about 1% to about 90%, or from about 5% to about 50% fabric softening active. The bluing agent may be present in the fabric care composition in an amount from about 0.5 ppb to about 50 ppm, or from about 0.5 ppm to about 30 ppm.

### Suitable Laundry Care Ingredients

While not essential for the purposes of the present invention, the non-limiting list of laundry care ingredients illustrated hereinafter are suitable for use in the laundry care compositions and may be desirably incorporated in certain aspects of the invention, for example to assist or enhance performance, for treatment of the substrate to be cleaned, or to modify the aesthetics of the composition as is the case with perfumes, colorants, dyes or the like. It is understood that such ingredients are in addition to the components that were previously listed for any particular aspect. The total amount of such adjuncts may range, once the amount of dye is taken into consideration from about 90% to about 99.99999995% by weight of the laundry care composition.

The precise nature of these additional components, and levels of incorporation thereof, will depend on the physical form of the composition and the nature of the operation for which it is to be used. Suitable laundry care ingredients include, but are not limited to, fabric softening actives, polymers, for example cationic polymers, surfactants, builders, chelating agents, dye transfer inhibiting agents, dispersants, enzymes, and enzyme stabilizers, catalytic materials, organic and inorganic opacifiers, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the laundry care ingredients are not essential to the laundry care compositions. Thus, certain aspects of the compositions do not contain one or more of the following adjunct materials: fabric softening actives, bleach activators, surfactants, builders, chelating agents, dye transfer inhibiting agents, dispersants, enzymes, and enzyme stabilizers, catalytic metal complexes, polymeric dispersing agents, clay and soil removal/anti-redeposition agents, brighteners, suds suppressors, dyes, additional perfumes and perfume delivery systems, structure elasticizing agents, fabric softeners, carriers, hydrotropes, processing aids and/or pigments. However, when one or more adjuncts are present, such one or more adjuncts may be present as detailed below:

### Surfactants

Suitable anionic surfactants useful herein can comprise any of the conventional anionic surfactant types typically used in liquid detergent products. These include the alkyl benzene sulfonic acids and their salts as well as alkoxylated or non-alkoxylated alkyl sulfate materials.

Exemplary anionic surfactants are the alkali metal salts of C₁₀-C₁₆ alkyl benzene sulfonic acids, or C₁₁-C₁₄ alkyl benzene sulfonic acids. In one aspect, the alkyl group is linear and such linear alkyl benzene sulfonates are known as “LAS”. Alkyl benzene sulfonates, and particularly LAS, are well known in the art. Such surfactants and their preparation are described for example in U.S. Pat. Nos. 2,220,099 and 2,477,383. Especially useful are the sodium and potassium linear straight chain alkylbenzene sulfonates in which the average number of carbon atoms in the alkyl group is from about 11 to 14. Sodium C₁₁-C₁₄, e.g., C₁₂, LAS is a specific example of such surfactants.

Another exemplary type of anionic surfactant comprises ethoxylated alkyl sulfate surfactants. Such materials, also known as alkyl ether sulfates or alkyl polyethoxylate sulfates, are those which correspond to the formula: R′—O—(C₂H₄O)ₙ—SO₃M wherein R′ is a C₈-C₂₀ alkyl group, n is from about 1 to 20, and M is a salt-forming cation. In one aspect, R′ is C₁₀-C₁₈ alkyl, n is from about 1 to 15, and M is sodium, potassium, ammonium, alkylammonium, or alkanolammonium. In one aspect, R′ is a C₁₂-C₁₆, n is from about 1 to 6 or even from about 1 to about 3 and M is sodium, potassium, ammonium, alkylammonium, or alkanolammonium, and, in one embodiment, is sodium.

The alkyl ether sulfates will generally be used in the form of mixtures comprising varying R′ chain lengths and varying degrees of ethoxylation. Frequently such mixtures will inevitably also contain some non-ethoxylated alkyl sulfate materials, i.e., surfactants of the above ethoxylated alkyl sulfate formula wherein n=0. Non-ethoxylated alkyl sulfates may also be added separately to the compositions of this invention and used as or in any anionic surfactant component which may be present. Specific examples of non-alkoxylated, e.g., non-ethoxylated, alkyl ether sulfate surfactants are those produced by the sulfation of higher C₈-C₂₀ fatty alcohols. Conventional primary alkyl sulfate surfactants have the general formula: ROSO₃-M⁺ wherein R is typically a linear C₈-C₂₀ hydrocarbyl group, which may be straight chain or branched chain, and M is a water-solubilizing cation. In one aspect, R is a C₁₀-C₁₅ alkyl, and M is alkali metal, more specifically R is C₁₂-C₁₄ and M is sodium.

Specific, non-limiting examples of anionic surfactants useful herein include: a) C₁₁-C₁₈ alkyl benzene sulfonates (LAS); b) C₁₀-C₂₀ primary and branched-chain alkyl sulfates (AS); c) C₁₀-C₁₈ secondary (2,3) alkyl sulfates having suitable cations including sodium, potassium, ammonium, and mixtures thereof; d) C₁₀-C₁₈ alkyl alkoxy sulfates (AEₓS) wherein x is from 1-30; e) C₁₀-C₁₈ alkyl alkoxy carboxylates in one aspect, comprising 1-5 ethoxy units; f) mid-chain branched alkyl sulfates as discussed in U.S. Pat. No. 6,020,303 and U.S. Pat. No. 6,060,443; g) mid-chain branched alkyl alkoxy sulfates as discussed in U.S. Pat. No. 6,008,181 and U.S. Pat. No. 6,020,303; h) modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548; i) methyl ester sulfonate (MES); and j) alpha-olefin sulfonate (AOS).

Suitable nonionic surfactants useful herein can comprise any of the conventional nonionic surfactant types typically used in liquid detergent products. These include alkoxylated fatty alcohols and amine oxide surfactants. In one aspect, for use in the liquid detergent products herein are those nonionic surfactants which are normally liquid.

Suitable nonionic surfactants for use herein include the alcohol alkoxylate nonionic surfactants. Alcohol alkoxylates are materials which correspond to the general formula: R¹(CₘH₂ₘO)ₙOH wherein R¹ is a C₈-C₁₆ alkyl group, m is from 2 to 4, and n ranges from about 2 to 12. In one aspect, R¹ is an alkyl group, which may be primary or secondary, that comprises from about 9 to 15 carbon atoms, from about 12 to 15 carbon atoms, or from about 10 to 14 carbon atoms. In one aspect, the alkoxylated fatty alcohols will also be ethoxylated materials that contain from about 2 to 12 ethylene oxide moieties per molecule, from about 3 to 10 ethylene oxide moieties per molecule, or from about 7 to 9 ethylene oxide moieties per molecule.

The alkoxylated fatty alcohol materials useful in the liquid detergent compositions herein will frequently have a hydrophilic-lipophilic balance (HLB) which ranges from about 3 to 17 from about 6 to 15, or from about 8 to 15. Alkoxylated fatty alcohol nonionic surfactants have been marketed under the tradenames Neodol and Dobanol by the Shell Chemical Company.

Another suitable type of nonionic surfactant useful herein comprises the amine oxide surfactants. Amine oxides are materials which are often referred to in the art as “semi-polar” nonionics. Amine oxides have the formula: R(EO)ₓ(PO)_{y}(BO)_{z}N(O)(CH₂R′)_{2′}qH₂O. In this formula, R is a relatively long-chain hydrocarbyl moiety which can be saturated or unsaturated, linear or branched, and can contain from 8 to 20, 10 to 16 carbon atoms, or is a C₁₂-C₁₆ primary alkyl. R′ is a short-chain moiety, in one aspect R′ may be selected from hydrogen, methyl and —CH₂OH. When x+y+z is different from 0, EO is ethyleneoxy, PO is propyleneneoxy and BO is butyleneoxy. Amine oxide surfactants are illustrated by C₁₂₋₁₄ alkyldimethyl amine oxide.

Non-limiting examples of nonionic surfactants include: a) C₁₂-C₁₈ alkyl ethoxylates, such as, NEODOL® nonionic surfactants from Shell; b) C₆-C₁₂ alkyl phenol alkoxylates wherein the alkoxylate units are a mixture of ethyleneoxy and propyleneoxy units; c) C₁₂-C₁₈ alcohol and C₆-C₁₂ alkyl phenol condensates with ethylene oxide/propylene oxide block polymers such as Pluronic® from BASF; d) C₁₄-C₂₂ mid-chain branched alcohols, BA, as discussed in U.S. Pat. No. 6,150,322; e) C₁₄-C₂₂ mid-chain branched alkyl alkoxylates, BAEₓ, wherein x if from 1-30, as discussed in U.S. Pat. No. 6,153,577, U.S. Pat. No. 6,020,303 and U.S. Pat. No. 6,093,856; f) Alkylpolysaccharides as discussed in U.S. Pat. No. 4,565,647 to Llenado, issued Jan. 26, 1986; specifically alkylpolyglycosides as discussed in U.S. Pat. No. 4,483,780 and U.S. Pat. No. 4,483,779; g) Polyhydroxy fatty acid amides as discussed in U.S. Pat. No. 5,332,528, WO 92/06162, WO 93/19146, WO 93/19038, and WO 94/09099; and h) ether capped poly(oxyalkylated) alcohol surfactants as discussed in U.S. Pat. No. 6,482,994 and WO 01/42408.

In the laundry detergent compositions herein, the detersive surfactant component may comprise combinations of anionic and nonionic surfactant materials. When this is the case, the weight ratio of anionic to nonionic will typically range from 10:90 to 90:10, more typically from 30:70 to 70:30.

Cationic surfactants are well known in the art and non-limiting examples of these include quaternary ammonium surfactants, which can have up to 26 carbon atoms. Additional examples include a) alkoxylate quaternary ammonium (AQA) surfactants as discussed in U.S. Pat. No. 6,136,769; b) dimethyl hydroxyethyl quaternary ammonium as discussed in U.S. Pat. No. 6,004,922; c) polyamine cationic surfactants as discussed in WO 98/35002, WO 98/35003, WO 98/35004, WO 98/35005, and WO 98/35006; d) cationic ester surfactants as discussed in U.S. Pat. Nos. 4,228,042, 4,239,660 4,260,529 and U.S. Pat. No. 6,022,844; and e) amino surfactants as discussed in U.S. Pat. No. 6,221,825 and WO 00/47708, specifically amido propyldimethyl amine (APA).

Non-limiting examples of zwitterionic surfactants include derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 to Laughlin et al., issued Dec. 30, 1975 at column 19, line 38 through column 22, line 48, for examples of zwitterionic surfactants; betaine, including alkyl dimethyl betaine and cocodimethyl amidopropyl betaine, C₈ to C₁₈ (in one aspect C₁₂ to C₁₈) amine oxides and sulfo and hydroxy betaines, such as N-alkyl-N,N-dimethylammino-1-propane sulfonate where the alkyl group can be C₈ to C₁₈, or C₁₀ to C₁₄.

Non-limiting examples of ampholytic surfactants include aliphatic derivatives of secondary or tertiary amines, or aliphatic derivatives of heterocyclic secondary and tertiary amines in which the aliphatic radical can be straight- or branched-chain. One of the aliphatic substituents comprises at least about 8 carbon atoms, typically from about 8 to about 18 carbon atoms, and at least one comprises an anionic water-solubilizing group, e.g. carboxy, sulfonate, sulfate. See U.S. Pat. No. 3,929,678 to Laughlin et al., issued Dec. 30, 1975 at column 19, lines 18-35, for examples of ampholytic surfactants.

### Aqueous, Non-Surface Active Liquid Carrier

As noted, the laundry care compositions may be in the form of a solid, either in tablet or particulate form, including, but not limited to particles, flakes, sheets, or the like, or the compositions may be in the form of a liquid. The liquid detergent compositions may comprise an aqueous, non-surface active liquid carrier. Generally, the amount of the aqueous, non-surface active liquid carrier employed in the compositions herein will be effective to solubilize, suspend or disperse the composition components. For example, the liquid detergent compositions may comprise, based on total liquid detergent composition weight, from about 5% to about 90%, from about 10% to about 70%, or from about 20% to about 70% of the aqueous, non-surface active liquid carrier.

The most cost effective type of aqueous, non-surface active liquid carrier is typically water. Accordingly, the aqueous, non-surface active liquid carrier component will generally be mostly, if not completely, comprised of water. While other types of water-miscible liquids, such alkanols, diols, other polyols, ethers, amines, and the like, have conventionally been added to liquid detergent compositions as co-solvents or stabilizers, for purposes of the present invention, the utilization of such water-miscible liquids typically is minimized to hold down composition cost. Accordingly, the aqueous liquid carrier component of the liquid detergent products herein will generally comprise water present in concentrations ranging from about 5% to about 90%, or from about 5% to about 70%, by weight of the liquid detergent composition.

### Bleaching Agents

The cleaning compositions of the present invention may comprise one or more bleaching agents. Suitable bleaching agents other than bleaching catalysts include photobleaches, bleach activators, hydrogen peroxide, sources of hydrogen peroxide, pre-formed peracids and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) photobleaches for example sulfonated zinc phthalocyanine;

(2) preformed peracids including, but are not limited to, compounds selected from the group consisting of percarboxylic acids and salts, percarbonic acids and salts, perimidic acids and salts, peroxymonosulfuric acids and salts, for example, Oxzone®, and mixtures thereof;

(3) sources of hydrogen peroxide, for example, inorganic perhydrate salts, including alkali metal salts such as sodium salts of perborate (usually mono- or tetra-hydrate), percarbonate, sulfatepersulfate, perphosphate, persilicate salts and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated.; and

(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Bleach Boosting Compounds—The compositions herein may comprise one or more bleach boosting compounds. Bleach boosting compounds provide increased bleaching effectiveness in lower temperature applications. The bleach boosters act in conjunction with conventional peroxygen bleaching sources to provide increased bleaching effectiveness. This is normally accomplished through in situ formation of an active oxygen transfer agent such as a dioxirane, an oxaziridine, or an oxaziridinium. Alternatively, preformed dioxiranes, oxaziridines and oxaziridiniums may be used.

Among suitable bleach boosting compounds for use in accordance with the present invention are cationic imines, zwitterionic imines, anionic imines and/or polyionic imines having a net charge of from about +3 to about −3, and mixtures thereof.

Suitable bleach boosting compounds include zwitterionic bleach boosters zwitterionic bleach boosters, which are described in U.S. Pat. Nos. 5,576,282 and 5,718,614. Other bleach boosting compounds include cationic bleach boosters described in U.S. Pat. Nos. 5,360,569; 5,442,066; 5,478,357; 5,370,826; 5,482,515; 5,550,256; and WO 95/13351, WO 95/13352, and WO 95/13353.

Peroxygen sources are well-known in the art and the peroxygen source employed in the present invention may comprise any of these well known sources, including peroxygen compounds as well as compounds, which under consumer use conditions, provide an effective amount of peroxygen in situ.

Enzyme Bleaching—Enzymatic systems may be used as bleaching agents. The hydrogen peroxide may also be present by adding an enzymatic system (i.e. an enzyme and a substrate therefore) which is capable of generating hydrogen peroxide at the beginning or during the washing and/or rinsing process. Such enzymatic systems are disclosed in EP Patent Application 91202655.6 filed Oct. 9, 1991.

The present invention compositions and methods may utilize alternative bleach systems such as ozone, chlorine dioxide and the like. Bleaching with ozone may be accomplished by introducing ozone-containing gas having ozone content from about 20 to about 300 g/m³ into the solution that is to contact the fabrics. The gas:liquid ratio in the solution should be maintained from about 1:2.5 to about 1:6. U.S. Pat. No. 5,346,588 describes a process for the utilization of ozone as an alternative to conventional bleach systems and is herein incorporated by reference.

Builders—The compositions of the present invention can comprise one or more detergent builders or builder systems. When present, the compositions will typically comprise at least about 1% builder, or from about 5% or 10% to about 80%, 50%, or even 30% by weight, of said builder.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

### Process of Making

The liquid detergent compositions are in the form of an aqueous solution or uniform dispersion or suspension of surfactant, bluing agent, and certain optional other ingredients, some of which may normally be in solid form, that have been combined with the normally liquid components of the composition, such as the liquid alcohol ethoxylate nonionic, the aqueous liquid carrier, and any other normally liquid optional ingredients. Such a solution, dispersion or suspension will be acceptably phase stable and will typically have a viscosity which ranges from about 100 to 600 cps, or from about 150 to 400 cps. For purposes of this invention, viscosity is measured with a Brookfield LVDV-II+ viscometer apparatus using a #21 spindle.

The liquid detergent compositions herein can be prepared by combining the components thereof in any convenient order and by mixing, e.g., agitating, the resulting component combination to form a phase stable liquid detergent composition. In a process for preparing such compositions, a liquid matrix is formed containing at least a major proportion, or even substantially all, of the liquid components, e.g., nonionic surfactant, the non-surface active liquid carriers and other optional liquid components, with the liquid components being thoroughly admixed by imparting shear agitation to this liquid combination. For example, rapid stirring with a mechanical stirrer may usefully be employed. While shear agitation is maintained, substantially all of any anionic surfactants and the solid form ingredients can be added. Agitation of the mixture is continued, and if necessary, can be increased at this point to form a solution or a uniform dispersion of insoluble solid phase particulates within the liquid phase. After some or all of the solid-form materials have been added to this agitated mixture, particles of any enzyme material to be included, e.g., enzyme prills, are incorporated. As a variation of the composition preparation procedure hereinbefore described, one or more of the solid components may be added to the agitated mixture as a solution or slurry of particles premixed with a minor portion of one or more of the liquid components. After addition of all of the composition components, agitation of the mixture is continued for a period of time sufficient to form compositions having the requisite viscosity and phase stability characteristics. Frequently this will involve agitation for a period of from about 30 to 60 minutes.

In one aspect of forming the liquid detergent compositions, the bluing agent is first combined with one or more liquid components to form a bluing agent premix, and this bluing agent premix is added to a composition formulation containing a substantial portion, for example more than 50% by weight, more specifically, more than 70% by weight, and yet more specifically, more than 90% by weight, of the balance of components of the laundry detergent composition. For example, in the methodology described above, both the bluing agent premix and the enzyme component are added at a final stage of component additions. In another aspect, the bluing agent is encapsulated prior to addition to the detergent composition, the encapsulated bluing agent is suspended in a structured liquid, and the suspension is added to a composition formulation containing a substantial portion of the balance of components of the laundry detergent composition.

As noted previously, the detergent compositions may be in a solid form. Suitable solid forms include tablets and particulate forms, for example, granular particles, flakes or sheets. Various techniques for forming detergent compositions in such solid forms are well known in the art and may be used herein. In one aspect, for example when the composition is in the form of a granular particle, the bluing agent is provided in particulate form, optionally including additional but not all components of the laundry detergent composition. The bluing agent particulate is combined with one or more additional particulates containing a balance of components of the laundry detergent composition. Further, the bluing agent, optionally including additional but not all components of the laundry detergent composition, may be provided in an encapsulated form, and the bluing agent encapsulate is combined with particulates containing a substantial balance of components of the laundry detergent composition.

The laundry compositions of this invention, prepared as hereinbefore described, can be used to form aqueous washing solutions for use in the laundering of fabrics. Generally, an effective amount of such compositions is added to water, for example in a conventional fabric laundering automatic washing machine, to form such aqueous laundering solutions. The aqueous washing solution so formed is then contacted, typically under agitation, with the fabrics to be laundered therewith. An effective amount of the liquid detergent compositions herein added to water to form aqueous laundering solutions can comprise amounts sufficient to form from about 500 to 7,000 ppm of composition in aqueous washing solution, or from about 1,000 to 3,000 ppm of the detergent compositions herein will be provided in aqueous washing solution.

### EXAMPLES

The following are examples of the syntheses of the dye compounds as described herein.

### Example 1

In example 1, (E)-1,3-bis(3-ammoniopropyl)-2-((1,4-diethyl-1,2,3,4-tetrahydro-quinoxalin-6-yl)diazenyl)-1H-imidazol-3-ium bromide dichloride is prepared from tetrahydroquinoxaline and 2-aminoimidazole.

### Synthesis of 1,4-diethyl-1,2,3,4-tetrahydroquinoxaline

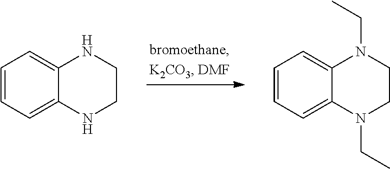

A mixture of tetrahydroquinoxaline (20.13 g), potassium carbonate (62.19 g) and bromoethane (49.04 g) in 120 mL of DMF is stirred at room temperature for 48 hours. The reaction mixture is filtered. Chromatography on silica gel yields 1,4-diethyl-1,2,3,4-tetrahydroquinoxaline as a yellow oil (16 g).

### Synthesis of (E)-6-((1H-imidazol-2-yl)diazenyl)-1,4-diethyl-1,2,3,4-tetrahydroquinoxaline

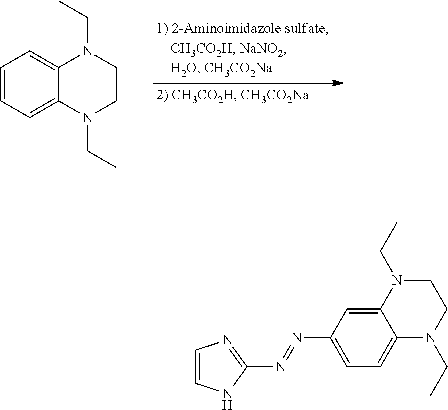 [SMILES: CCN1CCN(CC)c2ccccc21 ; CCN1CCN(CC)c2cc(/N=N/c3ncc[nH]3)ccc21]

2-Aminoimidazole sulfate (11.6 g) is dissolved in 7.4 mL of concentrated HCl, 8 mL of water, and 40 mL of acetic acid. The resulting solution is cooled to 0° C. To the solution is added a solution of 6.08 g of NaNO₂ in 16 mL of water dropwise where the internal temperature is maintained under 5° C. The resulting yellow-brown solution is stirred for 30 minutes at 0° C. In a separate flask equipped with a mechanical stirrer a mixture of 1,4-diethyl-1,2,3,4-tetrahydroquinoxaline, 13.1 g of sodium acetate and 40 mL of acetic acid is cooled to 0° C. To this slurry is added the diazonium solution slowly and the internal temperature of the reaction is maintained below 5° C. After the addition is complete, the resulting violet suspension is stirred for 1 hour at 0° C. The dark reaction mixture is poured into a large beaker containing 400 g ice. Aqueous NaOH (20%) is added to the suspension slowly until pH 6.5 is reached. The mixture is extracted with dichloromethane 6 times to yield crude (E)-6-((1H-imidazol-2-yl)diazenyl)-1,4-diethyl-1,2,3,4-tetrahydroquinoxaline (19.5 g, impure). This material is used in the next step without further purification.

### Synthesis of (E)-2-((1,4-diethyl-1,2,3,4-tetrahydroquinoxalin-6-yl)diazenyl)-1,3-bis(3-(2,2,2-trifluoroacetamido)propyl)-1H-imidazol-3-ium bromide

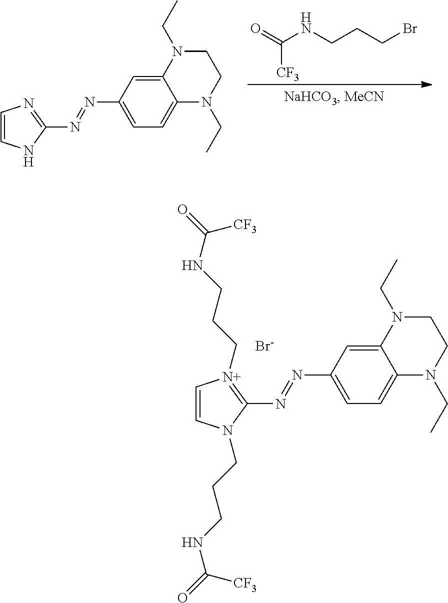 [SMILES: CCN1CCN(CC)c2cc(/N=N/c3ncc[nH]3)ccc21 ; O=C(NCCCBr)C(F)(F)F ; CCN1CCN(CC)c2cc(/N=N/c3n(CCCNC(=O)C(F)(F)F)cc[n+]3CCCNC(=O)C(F)(F)F)ccc21 ; [Br-]]

Crude (E)-6-((1H-imidazol-2-yl)diazenyl)-1,4-diethyl-1,2,3,4-tetrahydroquinoxaline (19.5 g) is dissolved in 150 mL of dry acetonitrile. NaHCO₃ (20.2 g) and 3-bromopropylamine trifluoroacetamide (37.4 g) are added and the mixture is stirred under reflux for 7 hours. The reaction mixture is filtered and concentrated. The resulting residue is purified by chromatography and triturated with cold acetone to yield pure (E)-2-((1,4-diethyl-1,2,3,4-tetrahydroquinoxalin-6-yl)diazenyl)-1,3-bis(3-(2,2,2-trifluoroacetamido)propyl)-1H-imidazol-3-ium bromide (9.6 g).

### Synthesis of (E)-1,3-bis(3-ammoniopropyl)-2-((1,4-diethyl-1,2,3,4-tetrahydroquinoxalin-6-yl)diazenyl)-1H-imidazol-3-ium bromide dichloride

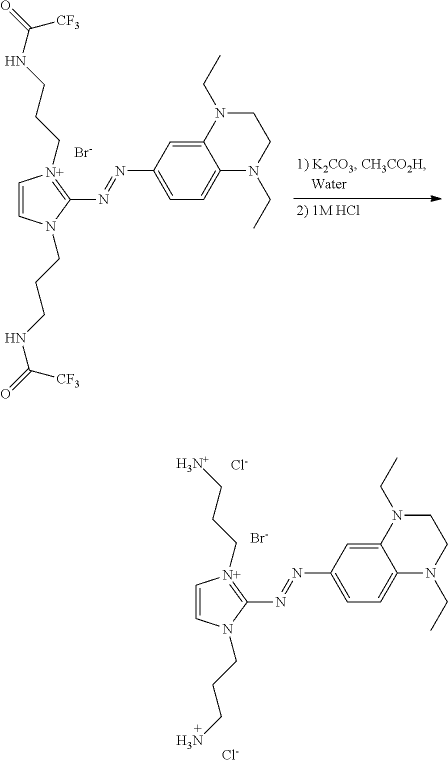 [SMILES: CCN1CCN(CC)c2cc(/N=N/c3n(CCCNC(=O)C(F)(F)F)cc[n+]3CCCNC(=O)C(F)(F)F)ccc21 ; [Br-] ; CCN1CCN(CC)c2cc(/N=N/c3n(CCCN)cc[n+]3CCC[NH3+])ccc21 ; [Cl-] ; [Br-] ; [Cl-]]

To a solution of 5.37 g (E)-2-((1,4-diethyl-1,2,3,4-tetrahydroquinoxalin-6-yl)diazenyl)-1,3-bis(3-(2,2,2-trifluoroacetamido)propyl)-1H-imidazol-3-ium bromide in 256 mL of ethanol is added 64 mL of 0.5 M K₂CO₃ solution. The resulting aqueous mixture is stirred at 40° C. for 24 hours. The reaction is cooled to room temperature and poured in to a large beaker. 1M HCl is added to adjust the solution to pH 7. Ethanol is removed by concentrating under vacuum at 35° C. The resulting aqueous dye solution is washed with dichloromethane 5 times and concentrated under reduced pressure to produce (E)-1,3-bis(3-ammoniopropyl)-2-((1,4-diethyl-1,2,3,4-tetrahydroquinoxalin-6-yl)diazenyl)-1H-imidazol-3-ium bromide dichloride.

### Example 2

In example 2, (E)-2-((1,4-diethyl-1,2,3,4-tetrahydroquinoxalin-6-yl)diazenyl)-1,3-bis(3-(trimethyl-ammonio)propyl)-1H-imidazol-3-ium tribromide is prepared from tetrahydroquinoxaline and 2-aminoimidazole.

### Synthesis of (E)-2-((1,4-diethyl-1,2,3,4-tetrahydroquinoxalin-6-yl)diazenyl)-1,3-bis(3-(trimethyl-ammonio)propyl)-1H-imidazol-3-ium tribromide

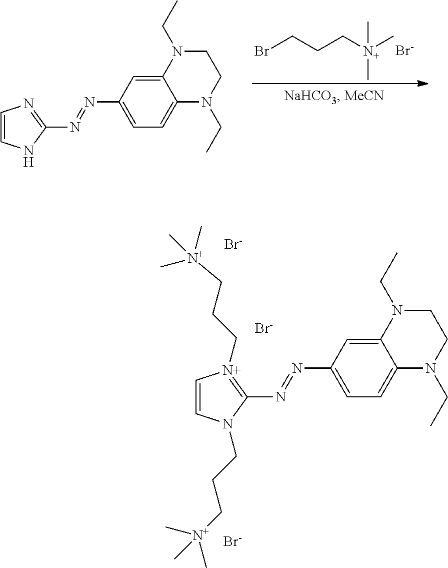

A mixture of (E)-6-((1H-imidazol-2-yl)diazenyl)-1,4-diethyl-1,2,3,4-tetrahydroquinoxaline (1 g), (3-bromopropyl)trimethyl ammonium bromide (2.75 g), and 886 mg of sodium bicarbonate in 10 mL acetonitrile is stirred under reflux for 5 hours. The reaction mixture is filtered and the dark solid collected is washed with acetonitrile and dried. The dark powder is then suspended in dry methanol and filtered to remove insoluble salt. The blue filtrate is concentrated under vacuum to yield 1.1 g of (E)-2-((1,4-diethyl-1,2,3,4-tetrahydroquinoxalin-6-yl)diazenyl)-1,3-bis(3-(trimethyl-ammonio)propyl)-1H-imidazol-3-ium tribromide.

### Example 3

In example 3 (E)-2-((1-methyl-4-(2,2,2-trifluoroacetyl)-1,2,3,4-tetrahydroquinoxalin-6-yl)-diazenyl)-1,3-bis(3-(2,2,2-trifluoroacetamido)propyl)-1H-imidazol-3-ium bromide is prepared from tetrahydroquinoxaline and 2-aminoimidazole.

### Synthesis of 1-(3,4-dihydroquinoxalin-1 (2H)-yl)-2,2,2-trifluoroethan-1-one

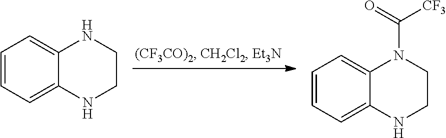 [SMILES: c1ccc2c(c1)NCCN2 ; O=C(N1CCNc2ccccc21)C(F)(F)F]

A round bottom flask is charged with 1.34 g of tetrahydroquinoxaline, 10 mL of dichloromethane and 1.11. g of trimethylamine. The reaction mixture is cooled in an ice bath and 1.94 g of trifluoroacetic anhydride is added dropwise. The resulting mixture is stirred at 0° C. for 30 minutes and then warmed up to room temperature. After filtration the solution obtained is dried and the residue is purified with silica gel chromatography. 1-(3,4-dihydroquinoxalin-1(2H)-yl)-2,2,2-trifluoroethan-1-one is obtained as white solid (1.01 g).

### Synthesis of 2,2,2-trifluoro-1-(4-methyl-3,4-dihydroquinoxalin-1(2H)-yl)ethan-1-one

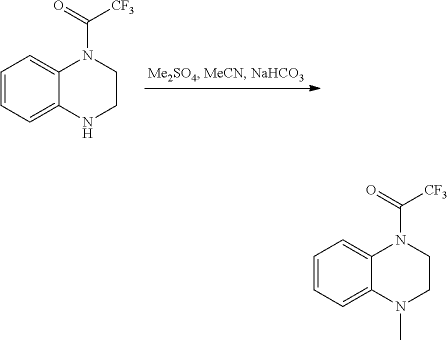 [SMILES: O=C(N1CCNc2ccccc21)C(F)(F)F ; CN1CCN(C(=O)C(F)(F)F)c2ccccc21]

To a mixture of 1-(3,4-dihydroquinoxalin-1(2H)-yl)-2,2,2-trifluoroethan-1-one (920 mg) and 672 mg of NaHCO₃ in 20 mL of acetonitrile is added 504 mg of dimethyl sulfate slowly. The mixture is heated at 85° C. for 10 hours. The crude material is filtered and concentrated, and the residue is purified by column chromatography to produce the 2,2,2-trifluoro-1-(4-methyl-3,4-dihydroquinoxalin-1(2H)-yl)ethan-1-one as yellow oil (820 mg).

### Synthesis of (E)-1-(7-((1H-imidazol-2-yl)diazenyl)-4-methyl-3,4-dihydroquinoxalin-1(2H)-yl)-2,2,2-trifluoroethan-1-one

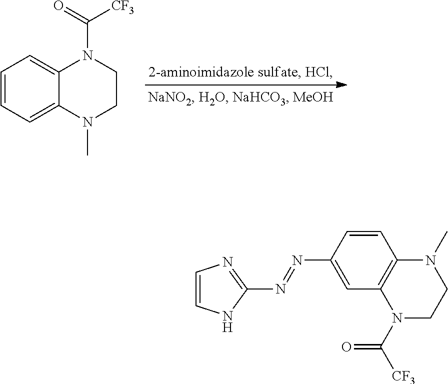 [SMILES: CN1CCN(C(=O)C(F)(F)F)c2ccccc21 ; CN1CCN(C(=O)C(F)(F)F)c2cc(/N=N/c3ncc[nH]3)ccc21]

A solution of 158 mg of 2-aminoimidazole sulfate in 0.4 mL of concentrated HCl and 0.4 mL of water is cooled to 0° C. To this solution is added dropwise a solution of 82.8 mg of NaNO₂ in 0.6 mL of water. The resulting yellow solution is stirred at 0° C. for 30 minutes and is added slowly to a suspension of 244 mg of 2,2,2-trifluoro-1-(4-methyl-3,4-dihydroquinoxalin-1(2H)-yl)ethan-1-one and 504 mg of NaHCO₃ in 3 mL of MeOH. The reaction mixture turns red. After the addition is complete, the mixture is filtered and the orange solid collected is dried to yield the crude product, (E)-1-(7-((1H-imidazol-2-yl)diazenyl)-4-methyl-3,4-dihydroquinoxalin-1(2H)-yl)-2,2,2-trifluoroethan-1-one. This material is used in the next step without further purification.

### Synthesis of (E)-2-((1-methyl-4-(2,2,2-trifluoroacetyl)-1,2,3,4-tetrahydroquinoxalin-6-yl)-diazenyl)-1,3-bis(3-(2,2,2-trifluoroacetamido)propyl)-1H-imidazol-3-ium bromide

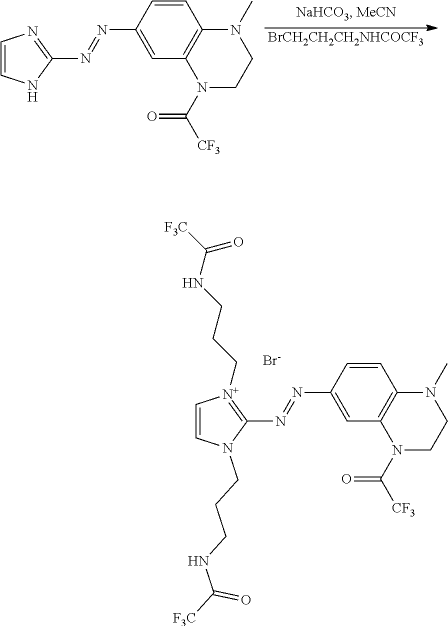

A mixture of crude (E)-1-(7-((1H-imidazol-2-yl)diazenyl)-4-methyl-3,4-dihydroquinoxalin-1(2H)-yl)-2,2,2-trifluoroethan-1-one 260 mg, 194 mg of NaHCO₃ and 540 mg of 3-bromopropylamine trifluoroacetamide is heated in 5 mL of MeCN at 86° C. for 16 hours. The reaction mixture is filtered and the filtrate is concentrated and the resulting residue is purified with column chromatography to yield 380 mg of (E)-2-((1-methyl-4-(2,2,2-trifluoroacetyl)-1,2,3,4-tetrahydroquinoxalin-6-yl)diazenyl)-1,3-bis(3-(2,2,2-trifluoroacetamido)-propyl)-1H-imidazol-3-ium bromide.

### Synthesis of (E)-1, 3-bis(3-ammoniopropyl)-2-((1-methyl-1,2,3,4-tetrahydroquinoxalin-6-yl)diazenyl)-1H-imidazol-3-ium bromide dichloride

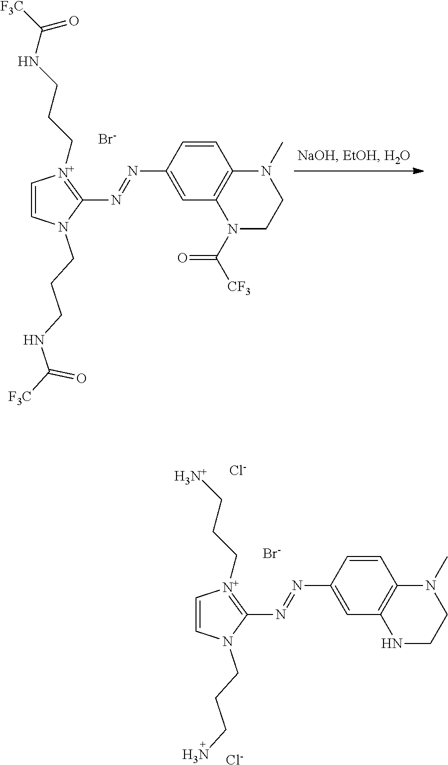 [SMILES: CN1CCN(C(=O)C(F)(F)F)c2cc(/N=N/c3n(CCCNC(=O)C(F)(F)F)cc[n+]3CCCNC(=O)C(F)(F)F)ccc21 ; [Br-] ; [Cl-] ; CN1CCNc2cc(/N=N/c3n(CCCN)cc[n+]3CCC[NH3+])ccc21 ; [Br-] ; [Cl-]]

A mixture of (E)-2-((1-methyl-4-(2,2,2-trifluoroacetyl)-1,2,3,4-tetrahydroquinoxalin-6-yl)diazenyl)-1,3-bis(3-(2,2,2-trifluoroacetamido)-propyl)-1H-imidazol-3-ium bromide (330 mg), 3.64 mL of 0.5 M NaOH and 14.5 mL of ethanol is stirred at room temperature until deprotection is complete. The pH of the reaction solution is adjusted to pH 7.0 with 1M HCl. The resulting solution is then concentrated under reduced pressure. (E)-1,3-bis(3-ammoniopropyl)-2-((1-methyl-1,2,3,4-tetrahydroquinoxalin-6-yl)diazenyl)-1H-imidazol-3-ium bromide dichloride is obtained as dark powder.

### Example 4

In example 4 (E)-1,3-bis(4-ammoniobutyl)-2-((1,4-dipentyl-1,2,3,4-tetrahydroquinoxalin-6-yl)diazenyl)-1H-imidazol-3-ium bromide dichloride is prepared from tetrahydroquinoxaline and 2-aminoimidazole.

### Synthesis of 1,4-dipentyl-1,2,3,4-tetrahydroquinoxaline

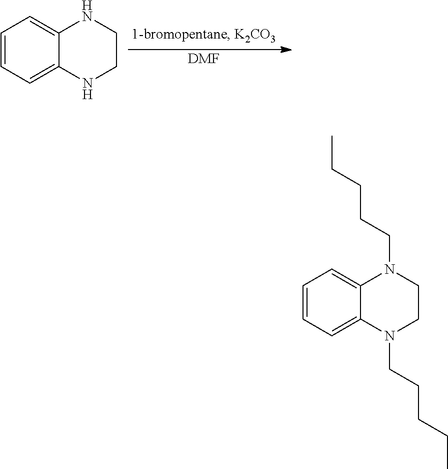

A suspension of tetrahydroquinoxaline (2.68 g), 1-bromopentane (9.06 g) and potassium carbonate (8.28 g) in 20 mL of DMF is stirred at room temperature for 3 days. The resulting mixture is filtered and the filtrate is loaded chromatographed on silica gel to give 1,4-dipentyl-1,2,3,4-tetrahydroquinoxaline (2.2 g) as yellow oil

### Synthesis of (E)-6-(1H-imidazol-2-yl)diazenyl)-1,4-dipentyl-1,2,3,4-tetrahydroquinoxaline

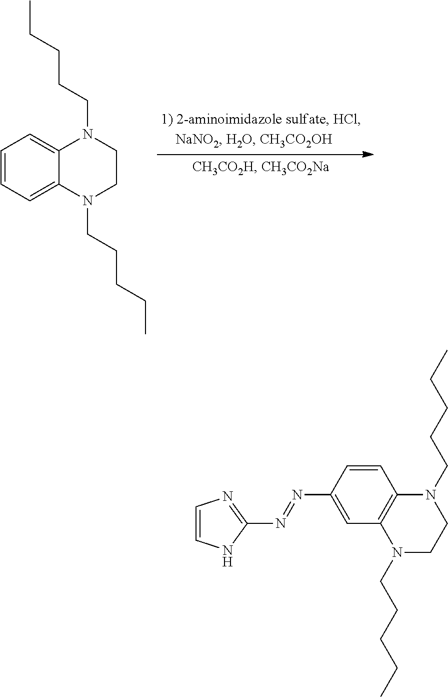 [SMILES: CCCCCN1CCN(CCCCC)c2ccccc21 ; CCCCCN1CCN(CCCCC)c2cc(/N=N/c3ncc[nH]3)ccc21]

2-Aminoimidazole sulfate (1.53 g) is dissolved in 0.97 mL of concentrated HCl, 1 mL of water, and 3 mL of acetic acid. The resulting solution is cooled to 0° C. A solution of 799 mg of NaNO₂ in 2 mL of water is added dropwise while the internal temperature is maintained under 5° C. The resulting yellow-brown solution is stirred for 30 minutes at 0° C. In a separate flask equipped with a mechanical stirrer a mixture of 1,4-dipentyl-1,2,3,4-tetrahydroquinoxaline (2.12 g), sodium acetate (1.9 g) and acetic acid (10 mL) is cooled to 0° C. To this slurry is added the diazonium solution slowly while stirring. After the addition is complete, the resulting red suspension is stirred for 1 hour at 0° C. The dark reaction mixture is poured into a large beaker containing 10 g ice. Aqueous NaOH (20%) is added to the suspension slowly until pH 6.5 is reached. The mixture is filtered and sticky dark gum is collected. This crude (E)-6-((1H-imidazol-2-yl)diazenyl)-1,4-dipentyl-1,2,3,4-tetrahydroquinoxaline is purified with column chromatography to give 0.9 g of pure product.

### Synthesis of N-(4-bromobutyl)-2,2,2-trifluoroacetamide

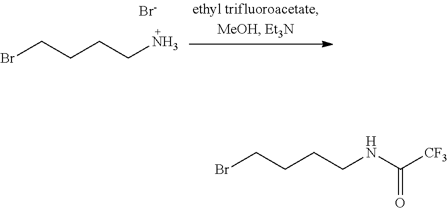 [SMILES: [Br-] ; NCCCCBr ; O=C(NCCCCBr)C(F)(F)F]

To a mixture of 4-bromo-1-butylamine (9.32 g) and ethyl trifluoroacetate (6.82 g) in 100 mL of methanol at 0° C. is added 6.06 g of trimethylamine dropwise. The reaction solution is warmed to room temperature and stirred overnight. The solvent is evaporated under vacuum and the residue is dissolved in methylene chloride. The solution is washed with water and 1M HCl and concentrated. Crude N-(4-bromobutyl)-2,2,2-trifluoroacetamide is obtained as colorless needles and is used in the next step without further purification.

### Synthesis of (E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium bromide

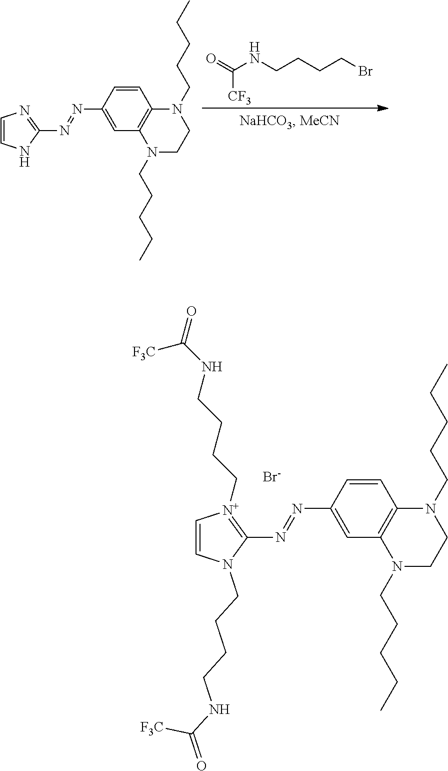

A mixture of crude (E)-6-((1H-imidazol-2-yl)diazenyl)-1,4-dipentyl-1,2,3,4-tetrahydroquinoxaline (630 mg), NaHCO₃ (431 mg) and 1.27 g of crude N-(4-bromobutyl)-2,2,2-trifluoroacetamide in 10 mL of MeCN is heated at 86° C. for 20 hours. The reaction is cooled to room temperature and filtered. The filtrate is concentrated and purified by column chromatography. (E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium bromide (790 mg) is obtained as dark solid.

### Synthesis of (E)-1,3-bis(4-ammoniobutyl)-2-((1,4-dipentyl-1,2,3,4-tetrahydroquinoxalin-6-yl)diazenyl)-1H-imidazol-3-ium bromide dichloride

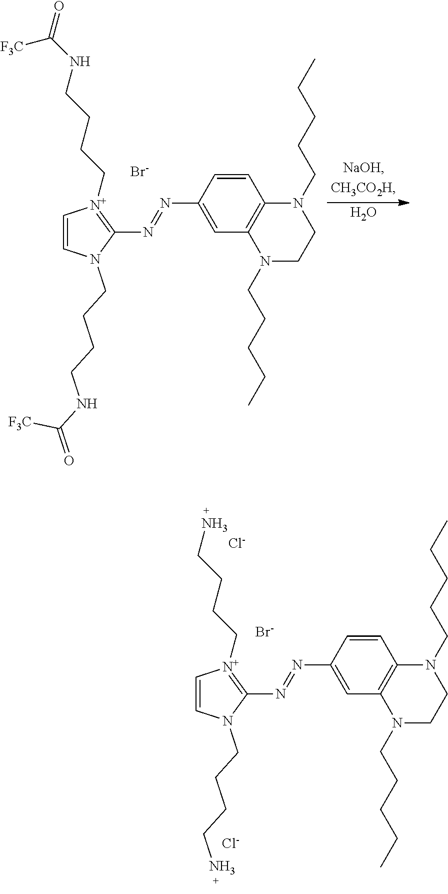

(E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium bromide (330 mg) is dissolved in 11.3 mL of ethanol and 2.81 mL of 0.5 M sodium hydroxide is added. The mixture is stirred at room temperature for 5 hours. The solution is adjusted to pH 7.0 by adding 1M HCl. The resulting solution is concentrated under vacuum and (E)-1,3-bis(4-ammoniobutyl)-2-((1,4-dipentyl-1,2,3,4-tetrahydroquinoxalin-6-yl)diazenyl)-1H-imidazol-3-ium bromide dichloride is obtained as a dark solid in quantitative yield.

### Example 5

In Example 5, (E)-1,3-bis(3-ammoniopropyl)-2-((1-pentyl-1,2,3,4-tetrahydro-quinoxalin-6-yl)diazenyl)-1H-imidazol-3-ium bromide dichloride is prepared from tetrahydroquinoxaline and 2-aminoimidazole.

### Synthesis of 1-pentyl-1,2,3,4-tetrahydroquinoxaline

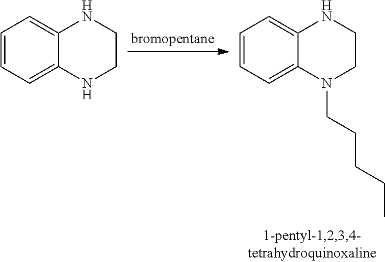

To a solution of 4.03 g of tetrahydroquinoxaline in 15 mL of DMF is added 4.53 g of 1-bromopentane. The mixture is stirred overnight at room temperature. The reaction mixture is chromatographed on silica gel column to give 1.35 g of pure 1-pentyl-1,2,3,4-tetrahydroquinoxaline.

### Synthesis of 2,2,2-trifluoro-1-(4-pentyl-3,4-dihydroquinoxalin-1(2H)-yl)ethan-1-one

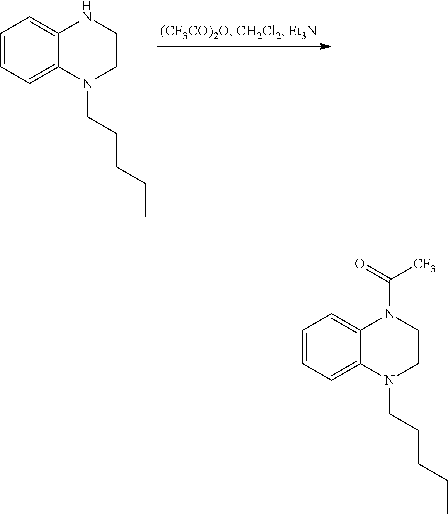 [SMILES: CCCCCN1CCNc2ccccc21 ; CCCCCN1CCN(C(=O)C(F)(F)F)c2ccccc21]

To a mixture of 1.22 g of 1-pentyl-1,2,3,4-tetrahydroquinoxaline and 1.21 g of Et₃N in 10 mL of dichloromethane at 0° C. is added 1.89 g of trifluoroacetic anhydride slowly. The mixture is warmed to room temperature and stirred overnight. Solvent is removed under reduced pressure and the residue is purified with column chromatography to produce 2,2,2-trifluoro-1-(4-pentyl-3,4-dihydroquinoxalin-1 (2H)-yl)ethan-1-one as yellow oil (1.3 g).

### Synthesis of (E)-1-(7-((1H-imidazol-2-yl)diazenyl)-4-pentyl-3,4-dihydroquinoxalin-1(2H)-yl)-2,2,2-trifluoroethan-1-one

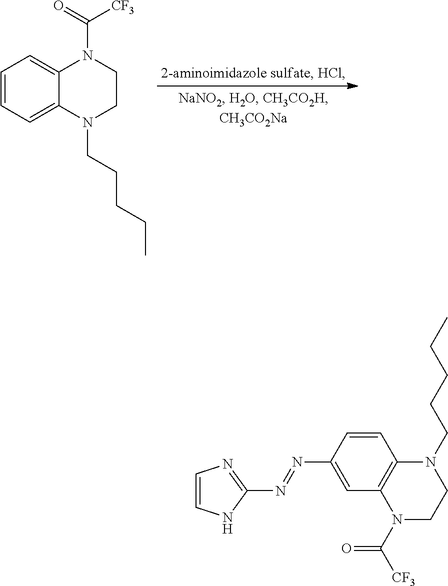 [SMILES: CCCCCN1CCN(C(=O)C(F)(F)F)c2ccccc21 ; CCCCCN1CCN(C(=O)C(F)(F)F)c2cc(/N=N/c3ncc[nH]3)ccc21]

2-Aminoimidazole sulfate (659 mg) is dissolved in 0.42 mL of concentrated HCl, 1 mL of water, and 3 mL of acetic acid. The resulting solution is cooled to 0° C. To the solution is added 345 mg of NaNO₂ in 1 mL of water dropwise so the internal temperature is maintained below 5° C. The resulting yellow-brown solution is stirred for 30 minutes at 0° C. In a separate flask equipped with a mechanical stirrer a mixture of 1.0 g of 2,2,2-trifluoro-1-(4-pentyl-3,4-dihydroquinoxalin-1(2H)-yl)ethan-1-one, 0.82 g of sodium acetate and 3 mL of acetic acid is cooled to 0° C. To this slurry is added the diazonium solution slowly while stirring. After the addition is complete, the resulting red suspension is stirred for 1 hour at 0° C. The dark reaction mixture is poured into a beaker containing 10 g of ice. Aqueous NaOH (20%) is added to the suspension slowly until pH 6.5 is reached. The mixture is filtered and brick red solid is collected. The crude (E)-1-(7-((1H-imidazol-2-yl)diazenyl)-4-pentyl-3,4-dihydroquinoxalin-1(2H)-yl)-2,2,2-trifluoroethan-1-one is dried and used in the next step without further purification (0.94 g).

### Synthesis of (E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium

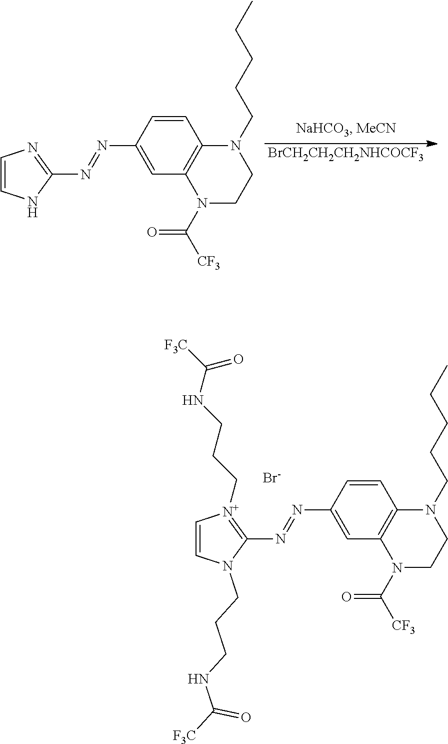

A mixture of 788 mg of (E)-1-(7-((1H-imidazol-2-yl)diazenyl)-4-pentyl-3,4-dihydroquinoxalin-1(2H)-yl)-2,2,2-trifluoroethan-1-one, 504 mg of NaHCO₃ and 1.4 g of N-(4-bromobutyl)-2,2,2-trifluoroacetamide in 12 mL of acetonitrile is heated at 86° C. for 20 hours. The reaction is cooled to room temperature and filtered. The filtrate is concentrated and purified with column chromatography. (E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium bromide is obtained as dark red solid (1.1 g).

### Synthesis of (E)-1,3-bis(3-ammoniopropyl)-2-((1-pentyl-1,2,3,4-tetrahydroquinoxalin-6-yl)diazenyl)-1H-imidazol-3-ium bromide dichloride

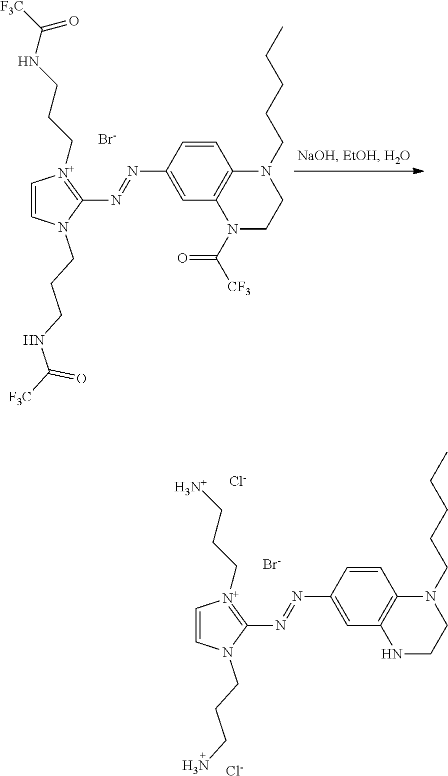 [SMILES: CCCCCN1CCN(C(=O)C(F)(F)F)c2cc(/N=N/c3n(CCCNC(=O)C(F)(F)F)cc[n+]3CCCNC(=O)C(F)(F)F)ccc21 ; [Br-] ; CCCCCN1CCNc2cc(/N=N/c3n(CCCN)cc[n+]3CCC[NH3+])ccc21 ; [Cl-] ; [Br-] ; [Cl-]]

A solution of 781 mg of (E)-2-((1,4-dipentyl-1,2,3,4-tetrahydroquinoxalin-6-yl)diazenyl)-1,3-bis(4-(2,2,2-trifluoroacetamido)butyl)-1H-imidazol-3-ium bromide is stirred in a mixture of 8 mL of 0.5M NaOH and 32 mL of ethanol for 5 hours at room temperature. The resulting blue solution is adjusted to pH 7.0 with 1M aqueous HCl. This solution is concentrated under vacuum to yield (E)-1,3-bis(3-ammoniopropyl)-2-((1-pentyl-1,2,3,4-tetrahydroquinoxalin-6-yl)diazenyl)-1H-imidazol-3-ium bromide dichloride as dark powder (quantitative).

### Example 6

In Example 6, (E)-1,3-bis(4-ammoniobutyl)-2-((1,4-diallyl-1,2,3,4-tetrahydroquinoxalin-6-yl)diazenyl)-1H-imidazol-3-ium bromide dichloride is prepared from tetrahydroquinoxaline and 2-aminoimidazole.

### Synthesis of 1,4-diallyl-1,2,3,4-tetrahydroquinoxaline

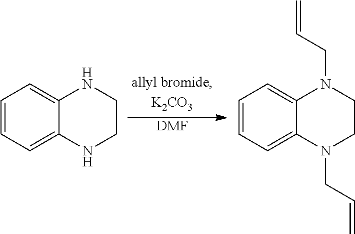

A suspension of 2.68 g of tetrahydroquinoxaline, 7.26 g of allyl bromide and 8.28 g of potassium carbonate in 20 mL DMF is stirred at room temperature for 3 days. The resulting mixture is filtered and the filtrate is chromatographed on silica gel. 1,4-Diallyl-1,2,3,4-tetrahydroquinoxaline is obtained as yellow oil (1.89 g).

### Synthesis of (E)-6-(1H-imidazol-2-yl)diazenyl)-1,4-diallyl-1,2,3,4-tetrahydroquinoxaline

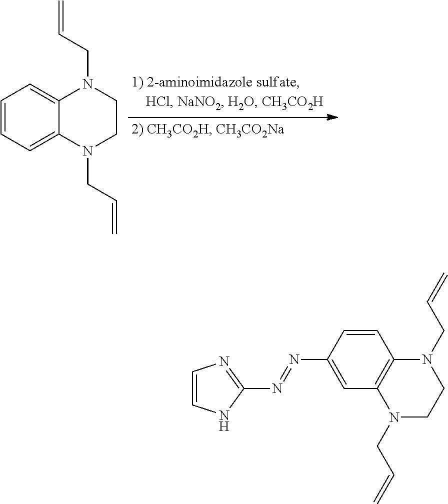 [SMILES: C=CCN1CCN(CC=C)c2ccccc21 ; C=CCN1CCN(CC=C)c2cc(/N=N/c3ncc[nH]3)ccc21]

2-Aminoimidazole sulfate (1.53 g) is dissolved in 0.97 mL of concentrated HCl, 1 mL of water, and 3 mL of acetic acid. The resulting solution is cooled to 0° C. A solution of 799 mg of NaNO₂ in 2 mL of water is added dropwise, and the internal temperature is maintained below 5° C. The resulting yellow-brown solution is stirred for 30 minutes at 0° C. In a separate flask equipped with a mechanical stirrer a mixture of 1.65 g of 1,4-diallyl-1,2,3,4-tetrahydroquinoxaline, 1.9 g of sodium acetate and 10 mL of acetic acid is cooled to 0° C. The diazonium solution is added slowly to this slurry while stirring. After the addition is complete, the resulting red suspension is stirred for 1 hour at 0° C. The dark reaction mixture is poured into a beaker containing 10 g of ice. Aqueous NaOH (20%) is added to the suspension slowly until pH 6.5 is reached. The mixture is filtered and the dark solid is dried. (E)-6-((1H-imidazol-2-yl)diazenyl)-1,4-diallyl-1,2,3,4-tetrahydroquinoxaline (2.3 g) is used in the next step without further purification.

### Synthesis of (E)-2-((1,4-diallyl-1,2,3,4-tetrahydroquinoxalin-6-yl)diazenyl)-1,3-bis(4-(2,2,2-trifluoroacetamido)butyl)-1H-imidazol-3-ium bromide

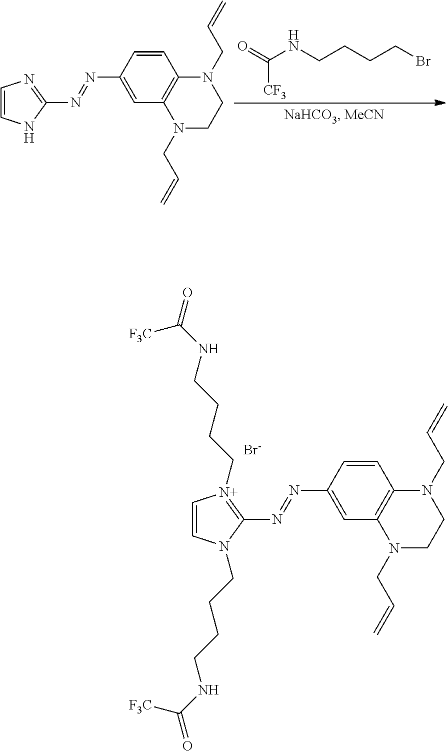

A mixture of 925 mg of (E)-6-((1H-imidazol-2-yl)diazenyl)-1,4-diallyl-1,2,3,4-tetrahydroquinoxaline, 756 mg of NaHCO₃ and 2.23 g of N-(4-bromobutyl)-2,2,2-trifluoroacetamide in 15 mL of acetonitrile is heated at 86° C. for 16 hours. The reaction is cooled to room temperature and filtered. The filtrate is concentrated and purified with column chromatography to obtain (E)-2-((1,4-diallyl-1,2,3,4-tetrahydroquinoxalin-6-yl)diazenyl)-1,3-bis(4-(2,2,2-trifluoroacetamido)butyl)-1H-imidazol-3-ium bromide as dark gum (0.85 g).

### Synthesis of (E)-1,3-bis(4-ammoniobutyl)-2-((1,4-diallyl-1,2,3,4-tetrahydroquinoxalin-6-yl)diazenyl)-1H-imidazol-3-ium bromide dichloride

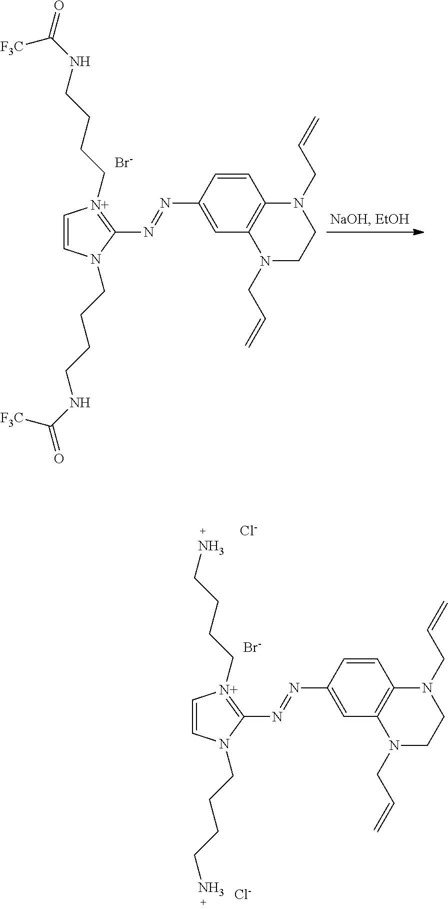 [SMILES: C=CCN1CCN(CC=C)c2cc(/N=N/c3n(CCCCNC(=O)C(F)(F)F)cc[n+]3CCCCNC(=O)C(F)(F)F)ccc21 ; [Br-] ; [Cl-] ; C=CCN1CCN(CC=C)c2cc(/N=N/c3n(CCCCN)cc[n+]3CCCCN)ccc21 ; [Br-] ; [Cl-]]

A mixture of (E)-2-((1,4-diallyl-1,2,3,4-tetrahydroquinoxalin-6-yl)diazenyl)-1,3-bis(4-(2,2,2-trifluoroacetamido)butyl)-1H-imidazol-3-ium bromide (450 mg) in 14.93 mL of ethanol and 3.73 mL of 0.5 M NaOH is stirred for 5 hours at room temperature. At completion of the reaction, 1 M HCl is added to lower the solution to pH 7. The solution is concentrated under vacuum and (E)-1,3-bis(4-ammoniobutyl)-2,4-diallyl-1,2,3,4-tetrahydroquinoxalin-6-yl)diazenyl)-1H-imidazol-3-ium bromide dichloride is obtained as a dark material (quantitative).

### Example 7

In Example 7, (E)-2-((1,4-diethyl-1,2,3,4-tetrahydroquinoxalin-6-yl)diazenyl)-1,3-dimethyl-1H-imidazol-3-ium methyl sulfate is prepared from quinoxaline and 2-aminoimidazole.

### Synthesis of 1,4-diethyl-1,2,3,4-tetrahydroquinoxaline

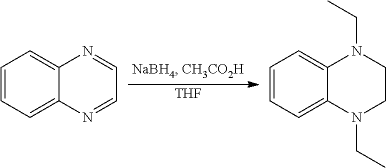

A round bottom flask charged with quinoxaline (1.95 g) and dry 30 mL of THF is cooled to 0° C. To the mixture is added 6 g of sodium borohydride over 15 minutes. The resulting slurry is stirred at 0° C. for 30 minutes and then 27 g of glacial acetic acid is added dropwise over 1 hour. The reaction temperature is maintained at 10° C. for 1 hour and the mixture is heated to reflux. After 16 hours, the reaction mixture is filtered and the filtrate is concentrated under vacuum. The crude 1,4-diethyl-1,2,3,4-tetrahydroquinoxaline is chromatographed on silica gel, dried and 1.55 g is used in the next steps without further purification.

### Synthesis of (E)-6-((1H-imidazol-2-yl)diazenyl)-1,4-diethyl-1,2,3,4-tetrahydroquinoxaline

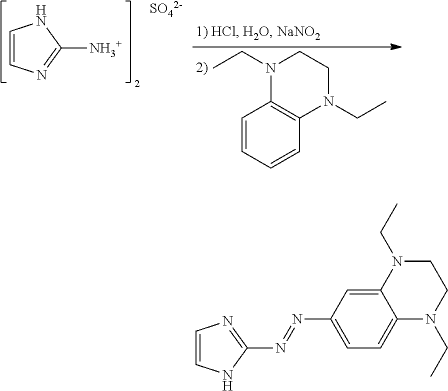

2-Aminoimidazole sulfate (145 mg) is dissolved in 0.34 mL of HCl and 0.4 mL of water and the resulting solution is cooled to 0° C. To the solution is added dropwise 76 mg of NaNO₂ in 1 mL of water. The resulting dark brown mixture is stirred for 30 minutes at 0° C. This mixture is then slowly added to a cooled solution of 190 mg of 1,4-diethyl-1,2,3,4-tetrahydroquinoxaline in 1 mL of methanol, keeping the temperature below 5° C. The resulting blue reaction mixture is stirred for 1 hour. Aqueous NaOH is added slowly until the mixture is pH 6.5. The reaction mixture is extracted with ethyl acetate to yield crude (E)-6-((1H-imidazol-2-yl)diazenyl)-1,4-diethyl-1,2,3,4-tetrahydroquinoxaline, which is used in the next step without further purification.

### Synthesis of (E)-2-((1,4-diethyl-1,2,3,4-tetrahydroquinoxalin-6-yl)diazenyl)-1,3-dimethyl-1H-imidazol-3-ium methyl sulfate

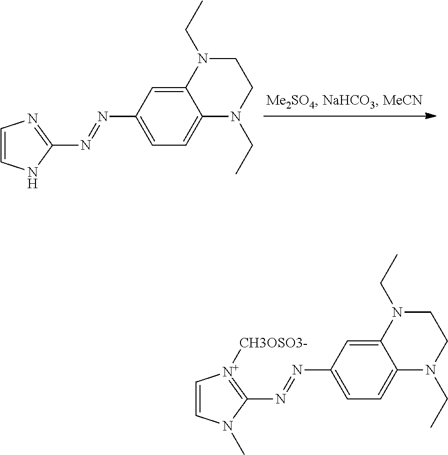

Crude (E)-6-((1H-imidazol-2-yl)diazenyl)-1,4-diethyl-1,2,3,4-tetrahydroquinoxaline is dissolved in 4 of mL dry acetonitrile. NaHCO₃ (126 mg) and 378 mg of dimethyl sulfate are added, and the mixture is stirred under reflux for 30 minutes. At completion, the reaction mixture is filtered and concentrated. The residue is purified with silica gel chromatography and (E)-2-((1,4-diethyl-1,2,3,4-tetrahydroquinoxalin-6-yl)diazenyl)-1,3-dimethyl-1H-imidazol-3-ium methyl sulfate is obtained as a blue solid (110 mg).

### Example 8

In Example 8, (E)-1,3-dimethyl-2-((2,3,4,5-tetrahydro-1,6-dioxa-3a-azaphenalen-8-yl)diazenyl)-1H-imidazol-3-ium methyl sulfate is prepared from 1,2,3-trifluoro-5-nitrobenzene and diethanolamine

### Synthesis of 8-nitro-2,3,4,5-tetrahydro-1,6-dioxa-3a-azaphenalene

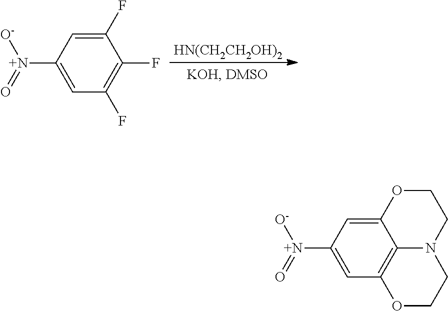

Diethanolamine (7 g) is dissolved in 50 mL of DMSO and 7 g of KOH is added. 3,4,5-Trifluoronitrobenzene (6 g) is added dropwise so that the temperature of the reaction mixture does not exceed 40° C. The reaction is stirred at 40° C. overnight. The mixture is then poured into 500 mL of ice water. The yellow-orange solid (2.1 g) is obtained by filtration is washed with water and dried to give 8-nitro-2,3,4,5-tetrahydro-1,6-dioxa-3a-azaphenalene, which is used in the next step without further purification.

### Synthesis of 2,3,4,5-tetrahydro-1,6-dioxa-3a-azaphenalen-8-amine

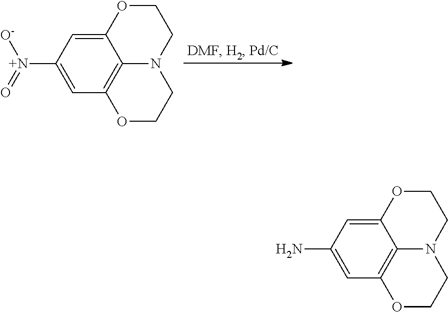

A dried pressure tube containing 111 mg of 8-nitro-2,3,4,5-tetrahydro-1,6-dioxa-3a-azaphenalene, 4 mL of DMF, and 11 mg of Pd/C is charged to 70 psi with hydrogen gas. The mixture is stirred overnight. To the reaction mixture is added 5 mL of 1 M HCl. The resulting solution is filtered and concentrated under vacuum. The 2,3,4,5-tetrahydro-1,6-dioxa-3a-azaphenalen-8-amine obtained is used in the next step without further purification.

### Synthesis of (E)-8-(1H-imidazol-2-yl)diazenyl)-2,3,4,5-tetrahydro-1,6-dioxa-3a-azaphenalene

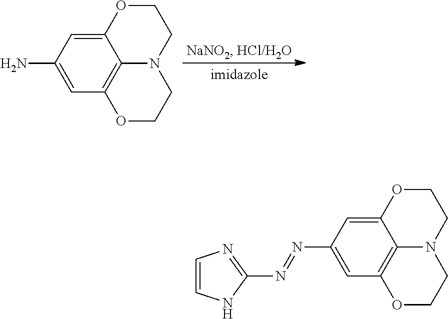

A solution of 96 mg of crude 2,3,4,5-tetrahydro-1,6-dioxa-3a-azaphenalen-8-amine from the previous step in 0.17 mL of concentrated HCl and 0.5 mL of water is cooled to 0° C. in an ice bath. To the mixture is added very slowly a solution of 38 mg of NaNO₂ in 0.5 mL of water while stirring. The reaction is allowed to continue for 30 minutes after complete addition of NaNO₂. Imidazole (37 mg) in 1 mL of water is added to the above solution slowly at 0° C., and the reaction is stirred for 1 hour. Aqueous 10% NaOH solution is added slowly to adjust the reaction to pH 8. The mixture is extracted with ethyl acetate and the organic layers are dried to yield crude (E)-8-((1H-imidazol-2-yl)diazenyl)-2,3,4,5-tetrahydro-1,6-dioxa-3a-azaphenalene, which is used in the next step without further purification.

### Synthesis of (E)-1,3-dimethyl-2-((2,3,4,5-tetrahydro-1,6-dioxa-3a-azaphenalen-8-yl)diazenyl)-1H-imidazol-3-ium methyl sulfate

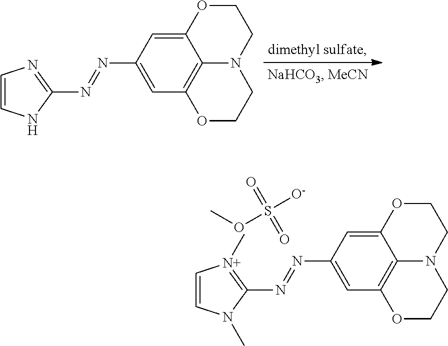

A solution of 135 mg of crude (E)-8-((1H-imidazol-2-yl)diazenyl)-2,3,4,5-tetrahydro-1,6-dioxa-3a-azaphenalene, 126 mg of NaHCO₃, and 142 mg of dimethyl sulfate in 5 mL of acetonitrile is heated under reflux for 4 hours. The reaction mixture is cooled to room temperature and filtered. The filtrate is concentrated under reduced pressure and the residue obtained is purified with column chromatography to produce (E)-1,3-dimethyl-2-((2,3,4,5-tetrahydro-1,6-dioxa-3a-azaphenalen-8-yl)diazenyl)-1H-imidazol-3-ium methyl sulfate as a dark solid (43 mg).

### Example 9

In Example 9, (E)-1,3-bis(4-ammoniobutyl)-2-((1,4-diallyl-1,2,3,4-tetrahydroquinoxalin-6-yl)diazenyl)-1H-imidazol-3-ium bromide dichloride is prepared from tetrahydroquinoxaline and 2-aminoimidazole.

### Synthesis of (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide

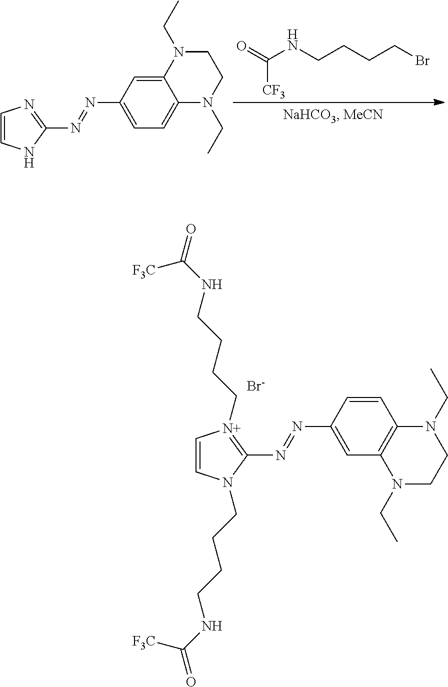

A mixture of 4-butylpropylamine trifluoroacetamide (20.00 g, 80.65 mmol), (E)-6-((1H-imidazol-2-yl)diazenyl)-1,4-diethyl-1,2,3,4-tetrahydroquinoxaline (7.50 g, 26.37 mmol) and NaHCO3 (4.90 g, 58.33 mmol) in acetonitrile (300 mL) is stirred at reflux under argon for 18 hours. After cooling to room temperature, additional NaHCO3 (500 mg, 5.95 mmol) and 2 (2.00 g, 8.06 mmol) are added. The reaction is stirred at reflux for 20 hours. The reaction mixture is cooled to room temperature, concentrated, and then purified by silica gel chromatography. The appropriate fractions are combined, concentrated and dried in vacuo at 40° C. The solids are washed with hexanes (2×50 mL) and re-dried in vacuo at 40° C. to give (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide as a dark blue film (12.3 g, 66.6% yield).

### Synthesis of (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide

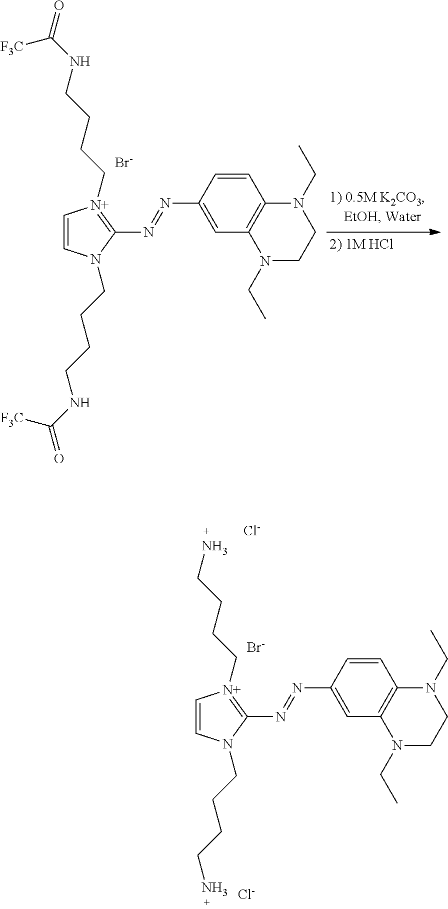 [SMILES: CCN1CCN(CC)c2cc(/N=N/c3n(CCCCNC(=O)C(F)(F)F)cc[n+]3CCCCNC(=O)C(F)(F)F)ccc21 ; [Br-] ; [Cl-] ; CCN1CCN(CC)c2cc(/N=N/c3n(CCCCN)cc[n+]3CCCCN)ccc21 ; [Br-] ; [Cl-]]

To a solution of (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide (505 mg, 0.722 mmol) in ethanol (24 mL) is added an aqueous solution of K₂CO₃ (400 mg, 2.89 mmol in 6 mL H2O). The reaction mixture is heated at 40° C. for 20 hours, cooled to RT, additional aqueous K₂CO₃ (100 mg, 0.72 mmol in 1 mL H₂O) is added. The reaction mixture is heated at 40° C. for 6 hours, cooled to room temperature and additional aq. K₂CO₃ (100 mg, 0.72 mmol in 1 mL H₂O) is added. The reaction mixture is heated at 40° C. for 16 hours, cooled to room temperature and carefully neutralized to pH 7.0 using 1N HCl (progress followed with a pH meter). Ethanol is removed in vacuo, and the residue is dissolved in water (25 mL). The aqueous solution of (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide is washed with CH₂Cl₂ (5×50 mL) then the water is removed in vacuo to give solid (E)-2-((1,4-diethyl-1,2,3,4-tetrahydroquinoxalin-6-yl)diazenyl)-1,3-bis(4-(2,2,2-trifluoroacetamido)butyl)-1H-imidazol-3-ium bromide.

### Example 10

In Example 10, Blue Polyethyleneimine Polymer is prepared from polyethyleneimine, tetrahydroquinoxaline, and 2-aminoimidazole.

### (E)-1,4-diethyl-6-((1-methyl-1H-imidazol-2-yl)diazenyl)-1,2,3,4-tetrahydroquinoxaline

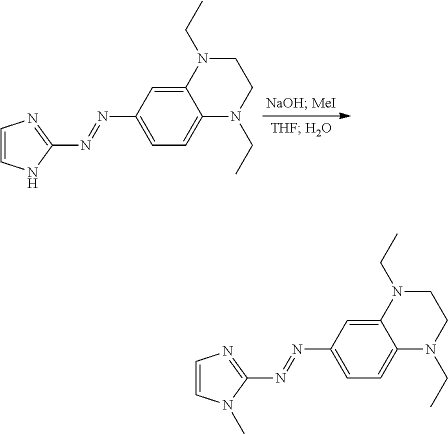 [SMILES: CCN1CCN(CC)c2cc(/N=N/c3ncc[nH]3)ccc21 ; CCN1CCN(CC)c2cc(/N=N/c3nccn3C)ccc21]

Crude (E)-6-((1H-imidazol-2-yl)diazenyl)-1,4-diethyl-1,2,3,4-tetrahydroquinoxaline (600 mg) is dissolved in 5 mL of THF and the suspension is cooled to 0° C. 40% NaOH solution (230 mg) is added slowly. The mixture is stirred for another 1-2 hours at 0-5° C. after is complete. MeI (327 mg) is added to the mixture dropwise. Stirring is continued for another 1-2 hours at 0-5° C. The reaction mixture is concentrated under vacuum to remove THF. The resulting aqueous solution is extracted with dichloromethane, concentrated and dried. The residue is purified by column chromatography to yield (E)-1,4-diethyl-6-((1-methyl-1H-imidazol-2-yl)diazenyl)-1,2,3,4-tetrahydroquinoxaline (550 mg).

### N—((E)-2-((1,4-diethyl-1,2,3,4-tetrahydroquinoxalin-6-yl)diazenyl)-1-methyl-3-pentyl-1H-imidazol-3-ium bromide)poly(ethylene)imine

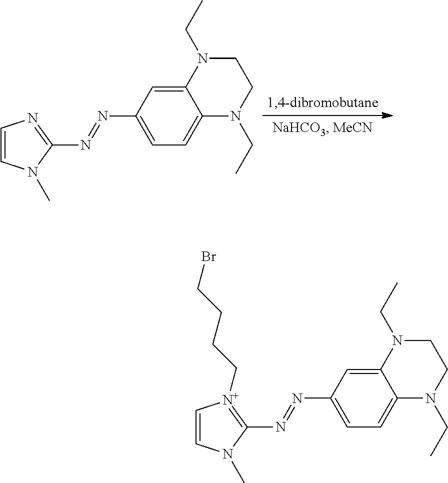 [SMILES: CCN1CCN(CC)c2cc(/N=N/c3nccn3C)ccc21 ; CCN1CCN(CC)c2cc(/N=N/c3n(C)cc[n+]3CCCCBr)ccc21]

(E)-1,4-diethyl-6-((1-methyl-1H-imidazol-2-yl)diazenyl)-1,2,3,4-tetrahydroquinoxaline (550 mg) is dissolved in 10 mL of MeCN, and NaHCO₃ (233 mg) and 1,4-dibromobutane (1.56 g) is added. After refluxing for 5 hours, the reaction mixture is cooled to room temperature and filtered. The filtrate is concentrated under vacuum and the residue is purified by silica gel chromatography to give (E)-3-(4-bromobutyl)-2-((1,4-diethyl-1,2,3,4-tetrahydroquinoxalin-6-yl)diazenyl)-1-methyl-1H-imidazol-3-ium as a dark powder (610 mg).

### Blue Polyethyleneimine Polymer

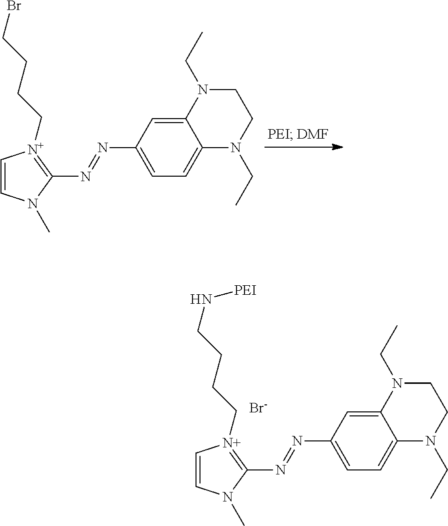

A mixture of [poly(ethylene)imine] (PEI) (126 mg) and (E)-3-(4-bromobutyl)-2-((1,4-diethyl-1,2,3,4-tetrahydroquinoxalin-6-yl)diazenyl)-1-methyl-1H-imidazol-3-ium 153 mg) in 5 mL of DMF is stirred for 96 hours at room temperature. The solvent is removed under vacuum, and the residue is triturated in dry acetone to give the final blue polyethyleneamine polymer.

### Exemplary Dye Formulations

% by weight

Composition A

| | |
|---|---|
| Stable Direct Blue Dye¹ | 0.001-2.0 |
| Ammonium Hydroxide (aq. 28% active) | 4.50 |
| Water | q.s. to 100 |

Composition B

| | |
|---|---|
| Stable Direct Blue Dye¹ | 0.005-2.0 |
| Ammonium carbonate | 10.00 |
| Water | q.s. to 100 |

Composition C

| | |
|---|---|
| Stable Direct Blue Dye¹ | 0.001-2.0 |
| FlexiThix ™ ³ | 5.00 |
| Phenoxyethanol | 0.30 |
| Sodium Benzoate | 0.30 |
| Disodium EDTA | 0.10 |
| Ammonium Hydroxide (aq. 28% active) | 4.00 |
| Water | q.s. to 100 |

Composition D

| | |
|---|---|
| Stable Direct Blue Dye¹ | 0.001-2.0 |
| Aculyn ™ 46⁴ | 15.80 |
| Phenoxyethanol | 0.30 |
| Sodium Benzoate | 0.30 |
| Disodium EDTA | 0.10 |
| Ammonium Hydroxide (aq. 28% active) | 4.00 |
| Water | q.s. to 100 |

Composition E

| | |
|---|---|
| Stable Direct Blue Dye¹ | 0.001-2.0 |
| Plantaren ® 2000 N UP² | 20.00 |
| Phenoxyethanol | 0.30 |
| Sodium Benzoate | 0.30 |
| Disodium EDTA | 0.10 |
| Ammonium Hydroxide (aq. 28% active) | 4.00 |
| Water | q.s. to 100 |

Composition F

| | |
|---|---|
| Stable Direct Blue Dye¹ | 0.001-2.0 |
| Non-anionic foaming agent | 5.00 |
| Phenoxyethanol | 0.30 |
| Sodium Benzoate | 0.30 |
| Disodium EDTA | 0.10 |
| Ammonium Hydroxide (aq. 28% active) | 4.00 |
| Water | q.s. to 100 |

¹The stable direct blue dye may be any one of the compounds described herein
²Chemical makeup supplied by BASF
³ PVP polymer supplied by Ashland
⁴PEG-150/Stearyl/SMDI copolymer supplied by Rohm and Haas

### Data

### In-Use Disappearance

Disappearance of the dye is measured on a Cary 100 UV/visible spectrophotometer at 25° C. A solution of the dye in water at a concentration sufficient to give an absorbance of 2.0 at the dye's λmax and buffered at pH 10.6 with ammonia is mixed with an equal volume of 6% hydrogen peroxide in a 1 cm quartz cuvette. The absorbance is measured at the λmax at zero (A₀) and thirty minutes (A₃₀) reaction time. Percent disappearance is calculated as the difference between the starting and thirty-minute absorbances divided by the starting absorbance minus the baseline absorbance (A_{b}), by the following equation:

$$\% Disappearance = \frac{( {A}_{0} - {A}_{30} )}{( {A}_{0} - {A}_{b} )} \times 100$$

Results are reported in Table 1 below.

Storage Stability

Disappearance of the dye is measured on a Cary 100 UV/visible spectrophotometer using a 1 cm cuvette. A solution of the dye is prepared at a concentration sufficient to give an absorbance of 0.9-1.1 at the dye's λmax when buffered at pH 10.0. The absorbance is measured after storage at 25° C. Percent disappearance is calculated as the difference between the starting absorbance (A₀) and the absorbance (Aₜ) at a specific storage period divided by the starting absorbance minus the baseline absorbance (A_{b}), by the following equation:

$$\% Disappearance = \frac{( {A}_{0} - {A}_{t} )}{( {A}_{0} - {A}_{b} )} \times 100$$

Results are reported in Table 1 below.

Color Intensity

Exemplary formulations indicated above are formulated with violet-blue to blue imidazolium azo dyes. These are applied directly to fabric test cloth, and then rinsed with water after thirty minutes. The resulting color is measured using a colorimeter with D65 illumination and characterized by the L* value. An L* of 100 is considered white and L* of 0 is considered black, therefore the higher the L* value the lower the color intensity. The measured L* values for different compounds of the present invention are summarized in Table 2 below.

TABLE 1
Color and stability (storage and in-use) for example dyes.

| | | | % | % |
|---|---|---|---|---|
| Compound | | Color | Disappearance | Disappearance |
| Number | Structure | (λmax; nm) | (In-use)* | (Storage)** |
| 1 | 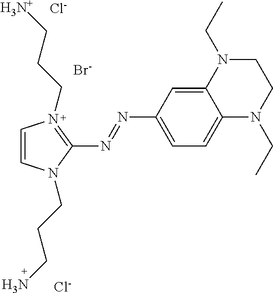 | 587 | 2.0 | 2.05 |
| 2 | 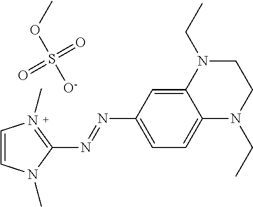 | 580 | 0 | 0.71 |
| 3 | 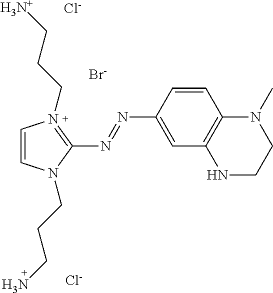 | 580 | 1.91 | 4.55 |
| 4 | 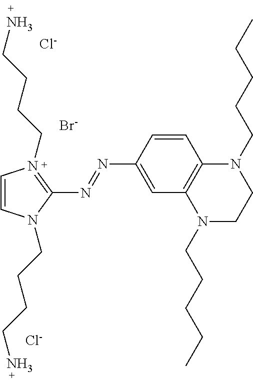 | 599 | 1.85 | 3.86 |
| 5 | 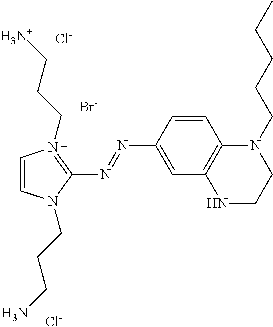 | 582 | 2.9 | 3.14 |
| 6 | 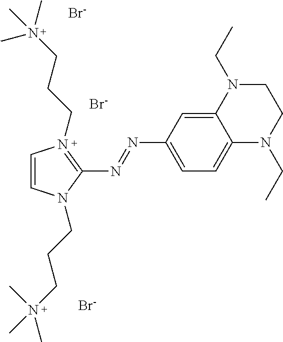 | 592 | 10.86 | 3.47 |
| 7 | 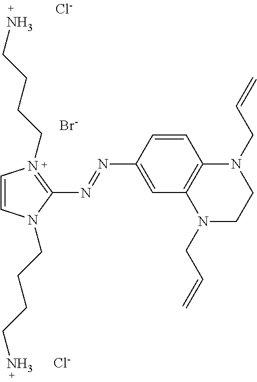 | 580 | 0.8 | 4.79 |
| 8 | 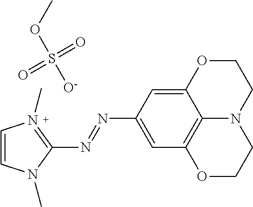 | 553 | 1.28 | 1.27 |
| 9 | 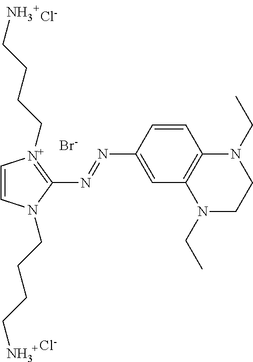 [SMILES: CCN1CCN(CC)c2cc(/N=N/c3n(CCCCN)cc[n+]3CCCCN)ccc21 ; [Cl-] ; [Cl-] ; [Br-]] | 587 | 1.85 | 3.37 |

*% loss recorded at 30 minutes in the presence of a 3% peroxide solution at pH 10.58, 25° C.
**% loss on 1-month storage at pH 10, 25° C.

TABLE 2
Colorstrike from imidazolium azo dyes in indicated composition.

| Compound | | Fabric/ | | | | | |
|---|---|---|---|---|---|---|---|
| Number | Structure | Composition¹ | L | a | b | C | h |
| 1 | 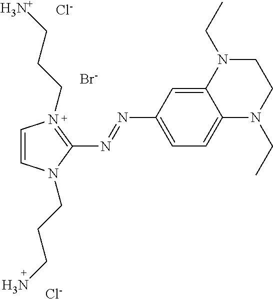 | Acetate/A Cotton/A Nylon/A Silk/A Viscose/A Wool/A | 81.02 64.33 63.48 23.94 53.76 35.54 | −3.43 −6.31 −8.12 −5.72 −7.17 −7.97 | −5.73 −12.26 −12.72 −17.49 −14.81 −14.96 | 6.68 13.79 15.09 18.4 16.46 16.95 | 239.06 242.76 237.45 251.88 244.17 241.96 |
| 2 | 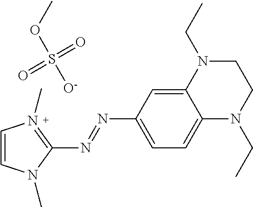 | Nylon/A Viscose/A | 42.24 46.66 | −7.16 −2.47 | −17.1 −18.15 | 18.54 18.32 | 247.29 262.24 |
| 4 | 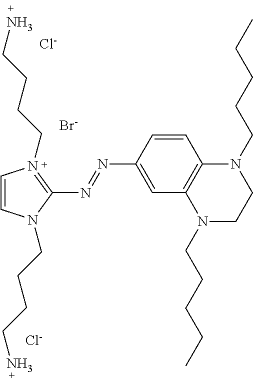 | Acetate/B Cotton/B Nylon/B Silk/B Viscose/B Wool/B | 73.49 49.64 77.87 29.19 49.02 49.46 | −4.49 −5.95 −7.08 −8.22 −5.91 −7.19 | −7.53 −14.49 −7.3 −17.51 −13.73 −9.97 | 8.77 15.66 10.17 19.34 14.95 12.29 | 239.19 247.68 225.87 244.85 246.72 234.19 |
| 5 | 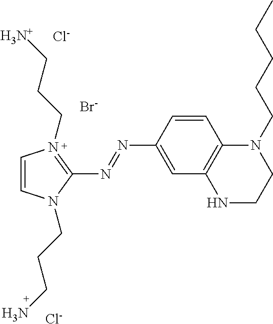 | Nylon/B Wool/B | 66.8 51.55 | −4.23 −3.18 | −12.04 −13.48 | 12.76 13.85 | 250.66 256.71 |
| 6 | 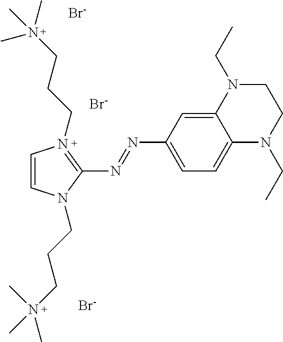 | Acetate/B Cotton/B Silk/B Viscose/B Wool/B | 85.1 57.48 37.4 53.68 56.8 | −1.24 −5.9 −8.45 −5.09 −6.17 | −3.05 −16.25 −18.61 −14.43 −8.26 | 3.29 17.29 20.44 15.3 10.31 | 247.86 250.05 245.57 250.58 233.25 |
| 9 | 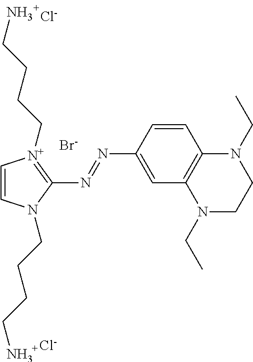 [SMILES: CCN1CCN(CC)c2cc(/N=N/c3n(CCCCN)cc[n+]3CCCCN)ccc21 ; [Cl-] ; [Cl-] ; [Br-]] | Acetate/B Cotton/B Nylon/B Silk/B Wool/B | 78.76 58.54 83.52 40.48 55.35 | −2.15 −5.1 −3.92 −8.44 −6.01 | −6.97 −15.88 −5.14 −21.68 −10.49 | 7.3 16.68 6.46 23.27 12.09 | 252.83 252.21 232.67 248.73 240.21 |
| 10¹ | 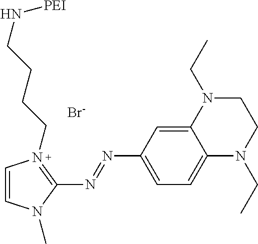 | Wool/C Viscose/C Silk/C Nylon/C Cotton/C Acetate/C | 56.68 55.21 49.03 71.37 51.49 68.14 | −5.71 −2.75 −8.72 −4.68 −4.8 −4.87 | −7.49 −15.02 −12.47 −7.7 −14.16 −9.61 | 9.42 15.27 15.22 9.01 14.95 10.78 | 232.71 259.63 235.03 238.72 251.27 243.11 |

¹Composition A: 4.5 g ammonia hydroxide (28%); dye; q.s. to 100 with water; pH = 10.82
Composition B: FlexiThix 5 g; Phenoxyethanol 0.3 g; sodium benzoate 0.3 g; sodium EDTA 0.1 g; dye; q.s. to 100 with water; pH = 6.98 with ammonium hydroxide
Composition C: dye; q.s. to 100 with water; pH 8.43

### Exemplary Detergent Formulations

### Formulations 1a to 1c—Liquid Detergent Formulations

Table 3 provides examples of liquid detergent formulations which include at least one of the compounds of the present invention.

TABLE 3
Liquid Detergent Formulations Comprising
the Inventive Compound

| | 1a | 1b | 1c |
|---|---|---|---|
| Ingredient | wt % | wt % | wt % |
| sodium alkyl ether sulfate | 14.4% | 14.4% | |
| linear alkylbenzene sulfonic acid | 4.4% | 4.4% | 12.2% |
| alkyl ethoxylate | 2.2% | 2.2% | 8.8% |
| amine oxide | 0.7% | 0.7% | 1.5% |
| citric acid | 2.0% | 2.0% | 3.4% |
| fatty acid | 3.0% | 3.0% | 8.3% |
| protease | 1.0% | 1.0% | 0.7% |
| amylase | 0.2% | 0.2% | 0.2% |
| borax | 1.5% | 1.5% | 2.4% |
| calcium and sodium formate | 0.2% | 0.2% | |
| amine ethoxylate polymers | 1.8% | 1.8% | 2.1% |
| DTPA¹ | 0.1% | 0.1% | |
| DTPMP² | | | 0.3% |
| fluorescent whitening agent | 0.15% | 0.15% | 0.2% |
| ethanol | 2.5% | 2.5% | 1.4% |
| propanediol | 6.6% | 6.6% | 4.9% |
| ethanolamine | 1.5% | 1.5% | 0.8% |
| sodium hydroxide | 3.0% | 3.0% | 4.9% |
| sodium cumene sulfonate | | | 2.0% |
| silicone suds suppressor | | | 0.01% |
| perfume | 0.3% | 0.3% | 0.7% |
| Non-tinting dyes³ | 0.0001% | 0.001% | 0.008% |
| Azo compound 1⁴ | | 0.001% | 0.001% |
| Azo compound 2⁴ | 0.003% | | 0.005% |
| water | balance | balance | balance |
| | 100.0% | 100.0% | 100.0% |

Footnotes for Formulations 1a-c:
¹diethylenetriaminepentaacetic acid, sodium salt
²diethylenetriaminepentakismethylenephosphonic acid, sodium salt
³a non-tinting dye or mixture of non-tinting dyes used to adjust formula color
⁴Azo compound selected from Table 2

### Formulations 2a-2c: Granular Detergent Formulations

Table 4 provides examples of granular detergent formulations which include at least one compound of the present invention. The formulations are shown in Table 4 as Formulations 2a through 2c.

TABLE 4
Granular Detergent Formulations
Comprising the Inventive Compound

| | 2a | 2b | 2c |
|---|---|---|---|
| Ingredient | wt % | wt % | wt % |
| Na linear alkylbenzene sulfonate | 3.4% | 3.3% | 11.0% |
| Na alkylsulfate | 4.0% | 4.1% | |
| Na alkyl sulfate (branched) | 9.4% | 9.6% | |
| alkyl ethoxylate | | | 3.5% |
| type A zeolite | 37.4% | 35.4% | 26.8% |
| sodium carbonate | 22.3% | 22.5% | 35.9% |
| sodium sulfate | 1.0% | | 18.8% |
| sodium silicate | | | 2.2% |
| protease | 0.1% | 0.2% | |
| sodium polyacrylate | 1.0% | 1.2% | 0.7% |
| carboxymethylcellulose | | | 0.1% |
| PEG 600 | | 0.5% | |
| PEG 4000 | | 2.2% | |
| DTPA | 0.7% | 0.6% | |
| fluorescent whitening agent | 0.1% | 0.1% | 0.1% |
| sodium percarbonate | | 5.0% | |
| sodium nonanoyloxybenzenesulfonate | | 5.3% | |
| silicone suds suppressor | 0.02% | 0.02% | |
| perfume | 0.3% | 0.3% | 0.2% |
| Azo compound 2¹ | 0.004% | | 0.001% |
| Azo compound 7¹ | | 0.006% | 0.002% |
| water and miscellaneous | balance | balance | Balance |
| | 100.0% | 100.0% | 100.0% |

¹Azo compounds selected from Table 2.

### Exemplary Fabric Care Compositions

### Formulations 3a-3c: Liquid Fabric Care Compositions

Table 5 provides examples of liquid fabric care compositions which include at least one compound of the present invention. The compositions are shown in Table 5 as Formulations 3a through 3c.

TABLE 5
Liquid Fabric Care Compositions
Comprising the Inventive Compound

| Ingredients | 3a | 3b | 3c |
|---|---|---|---|
| Fabric Softening Active ¹ | 13.70% | 13.70% | 13.70% |
| Ethanol | 2.14% | 2.14% | 2.14% |
| Cationic Starch ² | 2.17% | 2.17% | 2.17% |
| Perfume | 1.45% | 1.45% | 1.45% |
| Phase Stabilizing Polymer ³ | 0.21% | 0.21% | 0.21% |
| Calcium Chloride | 0.147% | 0.147% | 0.147% |
| DTPA ⁴ | 0.007% | 0.007% | 0.007% |
| Preservative ⁵ | 5 ppm | 5 ppm | 5 ppm |
| Antifoam ⁶ | 0.015% | 0.015% | 0.015% |
| Azo compound 1⁹ | 30 ppm | | 15 ppm |
| Azo compound 2⁹ | | 30 ppm | 15 ppm |
| Tinopal CBS-X ⁷ | 0.2 | 0.2 | 0.2 |
| Ethoquad C/25 ⁸ | 0.26 | 0.26 | 0.26 |
| Ammonium Chloride | 0.1% | 0.1% | 0.1% |
| Hydrochloric Acid | 0.012% | 0.012% | 0.012% |
| Deionized Water | Balance | Balance | Balance |

¹ N,N-di(tallowoyloxyethyl)-N,N-dimethylammonium chloride.
² Cationic starch based on common maize starch or potato starch, containing 25% to 95% amylose and a degree of substitution of from 0.02 to 0.09, and having a viscosity measured as Water Fluidity having a value from 50 to 84.
³ Copolymer of ethylene oxide and terephthalate having the formula described in U.S. Pat. No. 5,574,179 at col. 15, lines 1-5, wherein each X is methyl, each n is 40, u is 4, each R¹ is essentially 1,4-phenylene moieties, each R² is essentially ethylene, 1,2-propylene moieties, or mixtures thereof.
⁴ Diethylenetriaminepentaacetic acid.
⁵ KATHON ® CG available from Rohm and Haas Co.
⁶ Silicone antifoam agent available from Dow Corning Corp. under the trade name DC2310.
⁷ Disodium 4,4′-bis-(2-sulfostyryl) biphenyl, available from Ciba Specialty Chemicals.
⁸ Cocomethyl ethoxylated [15] ammonium chloride, available from Akzo Nobel.
⁹Azo compounds selected from Table 2.

It is noted that terms like “preferably,” “usually”, “generally,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is additionally noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound having a structure according to Formula (Xa), or a tautomer or salt thereof, 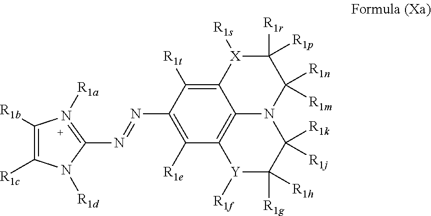
wherein
(i) R_{1g}, R₁ₕ, R₁ⱼ, R₁ₖ, R₁ₘ, R₁ₙ, R₁ₚ and R₁ᵣ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, nitro, nitroso, cyano, a heterocyclic moiety, thioether, thiol with or without a linker group, alkylsulfonate, alkylsulfate, carboxylalkyl, acrylamide or substituted acrylamides with or without a linker group, vinylsulfone with or without a linker group, sulfonyl ethyl sulfate with or without a linker group, halo-s-triazines with or without a linker group, halopyrimidines with or without a linker group, haloquinoxalines with or without a linker group, or are attached to a polymer backbone through a linker;
(ii) R_{1b}, R_{1c}, R₁ₑ and R₁ₜ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iii) R_{1f} and R₁ₛ are each independently hydrogen, alkyl, halogen substituted alkyl, alkenyl, alkynyl, aryl, hydroxyalkyl, aminoalkyl, alkyl group carrying a quaternary ammonium cation, acyl, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iv) R₁ₐ and R_{1d} are each independently alkyl, halogen substituted alkyl, alkenyl, alkynyl, aryl, hydroxyalkyl, aminoalkyl, alkyl group carrying a quaternary ammonium cation, alkyl group carrying a quaternary ammonium cation, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(v) X and Y are each independently an oxygen or a nitrogen atom and when X or Y is a nitrogen atom the other can be a carbon atom; wherein in the case where X and/or Y is oxygen atom, the corresponding group attached to the oxygen atom, R_{1f} and/or R_{1g}, ceases to exist.

2. A compound according to claim 1 wherein at least one of X and Y is an oxygen atom.

3. A compound according to claim 2 wherein X and Y are both oxygen atoms.

4. A compound according to claim 3 wherein R₁ₐ, R_{1d}, R_{1f} and R₁ₛ are each independently alkenyl, alkyl, aminoalkyl or alkyl group carrying a quaternary ammonium cation.

5. A compound according to claim 1, wherein the compound is attached to a polymer backbone through R₁ₐ, R_{1d}, R_{1f} or R₁ₛ.

6. A compound having a structure according to Formula (Xb), or a tautomer or salt thereof, 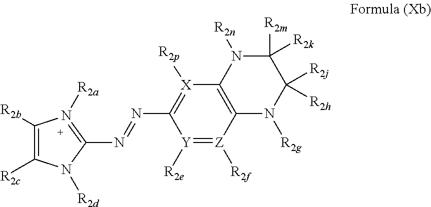
wherein
(i) R₂ₕ, R₂ⱼ, R₂ₖ and R₂ₘ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, nitro, nitroso, cyano, a heterocyclic moiety, thioether, thiol with or without a linker group, alkylsulfonate, alkylsulfate, carboxylalkyl, acrylamide or substituted acrylamides with or without a linker group, vinylsulfone with or without a linker group, sulfonyl ethyl sulfate with or without a linker group, halo-s-triazines with or without a linker group, halopyrimidines with or without a linker group, haloquinoxalines with or without a linker group, or are attached to a polymer backbone through a linker;
(ii) R_{2b}, R_{2c}, R₂ₑ, R_{2f} and R₂ₚ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iii) R_{2g} and R₂ₙ are each independently hydrogen, alkyl, halogen substituted alkyl, alkenyl, alkynyl, aryl, hydroxyalkyl, aminoalkyl, alkyl group carrying a quaternary ammonium cation, acyl, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker;
(iv) R₂ₐ and R_{2d} are each independently alkyl, halogen substituted alkyl, alkenyl, alkynyl, aryl, hydroxyalkyl, aminoalkyl, alkyl group carrying a quaternary ammonium cation, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(v) X, Y and Z are each independently a carbon or a nitrogen atom; wherein the total number of nitrogen atoms among X, Y and Z equals to 0 or 1; the total number of carbon atoms among X, Y and Z equals to 2 or 3; and, in the case where one of X, Y, Z is a nitrogen atom, the corresponding group attached to the nitrogen atom, one of R₂ₑ, R_{2f} or R₂ₚ, ceases to exist.

7. A compound according to claim 6, wherein R₂ₐ and R_{2d} are each independently alkyl, aminoalkyl or alkyl group carrying a quaternary ammonium cation.

8. A compound according to claim 7, wherein R₂ₐ is alkyl and R_{2d} is aminoalkyl or an alkyl group carrying a quaternary ammonium cation.

9. A compound according to claim 6, wherein R₂ₐ and R_{2d} are both an alkyl group carrying a quaternary ammonium cation.

10. A compound according to claim 6, wherein R_{2g} and R₂ₙ are each independently hydrogen, alkenyl, alkyl, aminoalkyl or an alkyl group carrying a quaternary ammonium cation.

11. A compound according to claim 10, wherein R_{2g} is hydrogen or alkyl and R₂ₙ is alkenyl or alkyl.

12. A compound according to claim 6, wherein the compound is attached to a polymer backbone through R₂ₐ, R_{2d}, R_{2g} or R₂ₙ.

13. A compound according to claim 6, wherein X, Y and Z are all carbon.

14. A compound according to claim 13, wherein R₂ₐ and R_{2d} are each independently alkyl, aminoalkyl, alkyl group carrying a quaternary ammonium cation; R_{2b}, R_{2c}, R₂ₑ, R_{2f} and R₂ₚ are each independently hydrogen or alkyl; R_{2g} and Rₙ are each independently hydrogen, alkenyl, aminoalkyl, alkyl group carrying a quaternary ammonium cation or alkyl; and R₂ₕ, R₂ⱼ, R₂ₖ and R₂ₘ are each independently hydrogen or alkyl.

15. A compound according to claim 14, wherein R_{2b}, R_{2c}, R₂ₑ, R_{2f}, R₂ₚ, R₂ₕ, R₂ⱼ and R₂ₖ, R₂ₘ are each hydrogen.

16. A compound having a structure according to Formula (Xb), or a tautomer or salt thereof, 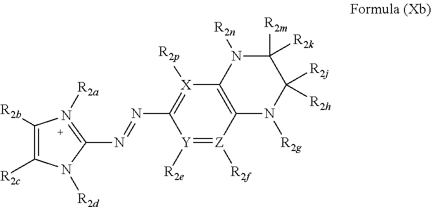
wherein
(i) R₂ₕ, R₂ⱼ and R₂ₖ, R₂ₘ are hydrogen;
(ii) R_{2b}, R_{2c}, R₂ₑ, R_{2f} and R₂ₚ are hydrogen;
(iii) R_{2g} and R₂ₙ are each independently hydrogen, alkenyl, alkyl, aminoalkyl or an alkyl group carrying a quaternary ammonium cation;
(iv) R₂ₐ and R_{2d} are each independently alkenyl, alkyl, aminoalkyl or an alkyl group carrying a quaternary ammonium cation; and
(v) X, Y and Z are carbon atoms.

17. A laundry care composition comprising a laundry care ingredient and from about 0.00001 wt % to about 0.5 wt % of a compound having a structure according to Formula (Xb), or a tautomer or salt thereof, 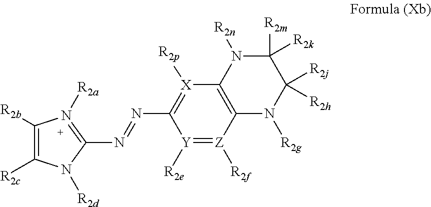
wherein
R₂ₕ, R₂ⱼ, R₂ₖ and R₂ₘ are each independently hydrogen, alkyl, halogen substituted alkyl, alkenyl, alkynyl, aryl, hydroxyalkyl, aminoalkyl, alkyl group carrying a quaternary ammonium cation, alkoxy, aryloxy, acyl, halogen, nitro, nitroso, cyano, a heterocyclic moiety, thioether, thiol with or without a linker group, alkylsulfonate, alkylsulfate, carboxylalkyl, acrylamide or substituted acrylamides with or without a linker group, vinylsulfone with or without a linker group, sulfonyl ethyl sulfate with or without a linker group, halo-s-triazines with or without a linker group, halopyrimidines with or without a linker group, haloquinoxalines with or without a linker group, or are attached to a polymer backbone through a linker; and
(ii) R_{2b}, R_{2c}, R₂ₑ, R_{2f} and R₂ₚ are each independently hydrogen, alkyl, halogen substituted alkyl, alkenyl, alkynyl, aryl, hydroxyalkyl, aminoalkyl, an alkyl group carrying a quaternary ammonium cation, alkoxy, aryloxy, acyl, halogen, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(iii) R_{2g} and R₂ₙ are each independently hydrogen, alkyl, halogen substituted alkyl, alkenyl, alkynyl, aryl, hydroxyalkyl, aminoalkyl, an alkyl group carrying a quaternary ammonium cation, acyl, a heterocyclic moiety, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(iv) R₂ₐ and R_{2d} are each independently alkyl, halogen substituted alkyl, alkenyl, alkynyl, aryl, hydroxyalkyl, aminoalkyl, an alkyl group carrying a quaternary ammonium cation, thioether, thiol with a linker group, alkylsulfonate, alkylsulfate, carboxylalkyl, acrylamide or substituted acrylamides with a linker group, vinylsulfone with a linker group, sulfonyl ethyl sulfate with a linker group, halo-s-triazines with a linker group, halopyrimidines with a linker group, haloquinoxalines with a linker group, or are attached to a polymer backbone through a linker; and
(v) X, Y and Z are each independently a carbon or a nitrogen atom; wherein the total number of nitrogen atoms among X, Y and Z equals to 0 or 1; the total number of carbon atoms among X, Y and Z equals to 2 or 3; and, in the case where one of X, Y, Z is a nitrogen atom, the corresponding group attached to the nitrogen atom, one of R₂ₑ, R_{2f} or R₂ₚ, ceases to exist.

18. A laundry care composition according to claim 17, wherein with respect to the compound of Formula (Xb),
(i) R₂ₕ, R₂ⱼ, R₂ₖ and R₂ₘ are hydrogen;
(ii) R_{2b}, R_{2c}, R₂ₑ, R_{2f} and R₂ₚ are hydrogen;
(iii) R_{2g} and R₂ₙ are each independently hydrogen, alkenyl, alkyl, aminoalkyl or an alkyl group carrying a quaternary ammonium cation;
(iv) R₂ₐ and R_{2d} are each independently alkenyl, alkyl, aminoalkyl or an alkyl group carrying a quaternary ammonium cation; and
(v) X, Y and Z are carbon atoms.

19. A laundry care composition according to claim 17 wherein the compound of Formula (Xb) is attached to a polymer selected from the group consisting of a linear polyethyleneimine; a branched polyethyleneimine consisting of primary, secondary and tertiary amine groups; a polyallylamine hydrochloride; a peptide, a protein; and mixtures thereof.

20. A laundry care composition according to claim 19 wherein the compound of Formula (Xb) is attached to a branched polyethyleneimine consisting of primary, secondary and tertiary amine groups.
